(19) United States
(12) Patent Application Publication
(10) Pub. No.: US 2024/0216913 A1
(43) Pub. Date: Jul. 4, 2024

(54) METHOD AND MICROFLUIDIC SYSTEM FOR THE ISOLATION OF PARTICLES

(71) Applicant: Menarini Silicon Biosystems S.p.A., Castel Maggiore (IT)

(72) Inventors: Gianni Medoro, Castel Maggiore (IT); Giulio Signorini, Castel Maggiore (IT)

(21) Appl. No.: 18/562,149

(22) PCT Filed: May 26, 2022
(86) PCT No.: PCT/IB2022/054960

§ 371 Date: Nov. 17, 2023

(30) Foreign Application Priority Data
May 26, 2021 (IT) 102021000013715

(51) Int. Cl. B01L 3/00; G01N 15/10; G01N 15/14; G01N 15/1433; G16B 40/10

(52) U.S. Cl. CPC B01L 3/502715; B01L 3/502761; G01N 15/1433; G01N 15/1484; G16B 40/10; B01L 2200/0652; B01L 2300/0663; B01L 2400/0487; G01N 2015/1006; G01N 2015/1027; G01N 2015/1497

(57) ABSTRACT

Method and microfluidic system (1) for the manipulation of particles; a detection device (7) acquires images of a specific particle (5) in a first position (IP) and in a second position (IIP); the difference is made between the two images in order to obtain a derived image in which the contours and the morphological characteristics of the specific particle (5) are more evident; in this manner, the type and the position of the particles can be identified more clearly, continuously and in a time-saving manner.

### CROSS-REFERENCE TO RELATED APPLICATIONS

This patent application claims priority from Italian patent application no. 102021000013715 filed on May 26, 2021, the entire disclosure of which is incorporated herein by reference.

### TECHNICAL FIELD OF THE INVENTION

The present invention relates to a method and microfluidic system for the manipulation and/or analysis of particles.

### BACKGROUND OF THE INVENTION

In the field of manipulation and/or analysis of particles, the microfluidic systems are known which comprise an inlet, through which, in use, the sample is inserted in the microfluidic system; and a moving assembly, which in turn comprises a microfluidic chamber and is adapted to move the particles inside the microfluidic chamber. Typically, the moving assembly comprises: a plurality of actuators, which are adapted to displace the particles; a detection device to acquire images of the microfluidic chamber; and a control device to control the actuators so as to move the particles inside the microfluidic chamber as a function of the images acquired by the detection device. Normally, the images are acquired by fluorescence in order to have a brighter representation of the shapes and/or of the positions of the particles.

This type of microfluidic systems has some drawbacks including as follows: the risk, in certain circumstances, of not being able to correctly identify and/or recognize some particles; of not being able to recover some particles; an operating speed that is not always optimal; the risk that some particles are damaged or contaminated.

Aim of the present invention is to provide a method and a microfluidic system for the manipulation and/or analysis of particles, which make it possible to overcome, at least partially, the drawbacks of the prior art and are, at the same time, easy and economical to implement.

### SUBJECT AND SUMMARY OF THE INVENTION

According to the present invention there are provided a method and a microfluidic system as set forth in the following independent claims and, preferably, in any of the claims directly or indirectly dependent on the independent claims.

Unless explicitly stated otherwise, the following terms have the meaning set forth hereinbelow in this text.

The equivalent diameter of a section is defined as the diameter of a circle having the same area as the section.

A microfluidic system is defined as a system comprising a microfluidic circuit, itself provided with at least one microfluidic channel and/or at least one microfluidic chamber. Advantageously but not necessarily, the microfluidic system comprises at least one valve (more in particular, a plurality of valves). Additionally or alternatively, the microfluidic system comprises at least one pump (more in particular, a plurality of pumps) and possibly at least one seal (more in particular, a plurality of seals).

In particular, a microfluidic channel is defined as a channel having a section with an equivalent diameter lower than 0.5 mm. In other words, a microfluidic channel has at least one stretch with section with equivalent diameter lower than 0.5 mm.

In particular, the microfluidic chamber has a height lower than 0.5 mm. More in particular, the microfluidic chamber has a width and length that are greater than the height (more precisely but not necessarily, at least five times the height).

A particle is defined as a corpuscle having the largest dimension lower than 500 μm (advantageously, lower than 150 μm; in particular, up to 40 μm; in particular, starting from 10 μm). According to some non-limiting examples, the particles are selected from: cells, cell debris (in particular, cell fragments; e.g., nuclei), exosomes, extracellular vesicles (such as, for example, extracellular vesicles of tumour origin), cell aggregates (such as, for example, small clusters of cells deriving from stem cells such as neurospheres or mammals), bacteria, lipospheres, micro-beads (in polystyrene and/or magnetic), nano-beads (e.g., nano-beads up to 100 nm,) complexes formed by micro-beads and/or nano-beads bound to cells (and a combination thereof). Advantageously, the particles are cells.

According to some non-limiting embodiments, the particles (advantageously cells and/or cell debris) have the largest dimension lower than 60 μm.

According to some specific, non-limiting embodiments, particles are chosen from the group consisting of: tumour cells, white blood cells (WBC), stromal cells, spermatozoa, circulating tumour cells (CTC), circulating myeloid cells (CMMC), nuclei, spores, foetal cells, micro-beads, liposomes, exosomes, extracellular vesicles (EV—e.g. extracellular vesicles of tumour origin—tdEVs), epithelial cells, erythroblasts, trophoblasts, erythrocytes, endothelial cells, stem cells (and combinations thereof).

Particle dimensions can be measured in a standard manner with graduated-scale microscopes or normal microscopes used with graduated-scale slides (on which the particles are deposited).

In this text, the dimensions of a particle is defined as the length, the width and the thickness of the particle. The term “in a substantially selective manner” is used to identify a displacement (or other similar terms indicating a movement) of particles relative to other particles (which typically do not move). In particular, the particles that are displaced and/or separated are mostly particles of one or more given types. Advantageously but not necessarily, a substantially selective displacement (or other analogous terms indicating a movement and/or separation) envisages displacing particles with at least 90% (advantageously 95%) of particles of the given type (s).

In this text, the expressions “downstream” and “upstream” are to be interpreted referring to the direction of the fluid flow and/or of the movement of the particles (from the inlet to an outlet of the microfluidic system).

In this text, when reference is made to a microfluidic system and/or a method for the manipulation and/or analysis of particles of a sample, it is not excluded that the sample comprises a single particle that is manipulated/analysed.

### BRIEF DESCRIPTION OF THE DRAWINGS

The invention will now be described with reference to the accompanying drawings, which show some non-limiting examples of embodiments, in which:

FIG. 1 schematically shows a system in accordance with the present invention;

FIG. 2 schematically shows a further (and more detailed) embodiment of the system of FIG. 1;

FIG. 3 schematically shows a detail of the operation of the system of FIG. 1 or 2 in two subsequent instants—t(0) and t(1);

FIG. 4 is a flowchart of an operating process of the system of FIG. 1 or 2;

FIG. 5 is a photograph taken in the visible range of a part of the system of FIG. 1 or 2;

FIG. 6 is an image showing what was obtained by using (according to n an operation mode with subtraction between subsequent photographs) the system of FIG. 1 or 2;

FIG. 7 shows parts of FIG. 6 on an enlarged scale;

FIG. 8 is a photograph showing what was obtained by using the system of FIG. 1 or 2 according to an operating mode (with difference between subsequent photographs) which is different from that implemented to obtain FIGS. 6 and 7;

FIG. 9 is a detail on enlarged scale of FIG. 5;

FIG. 10 is the detail of FIG. 9 in the subsequent instant;

FIG. 11 is an image obtained from the combination (difference) of FIGS. 9 and 10 (corresponds to a detail on an enlarged scale of FIG. 8);

FIG. 12 is an image obtained by inverting the image of FIG. 11;

FIG. 13 is a flowchart of an operating process of the system of FIG. 1 or 2;

FIGS. 14 and 15 are photographs showing the operating process shown by the flowchart of FIG. 13;

FIGS. 16 to 18 are flowcharts of respective operating processes of the system of FIG. 1 or 2;

FIG. 19 is a photograph used to verify the correct results obtained by the system of FIG. 1 or 2 operating according to the operating process of FIG. 18;

FIGS. 20 to 22 are flow diagrams of respective operating processes of the system of FIG. 1 or 2;

FIG. 23 is a block diagram showing schematically the operation of a neural network;

FIG. 24 schematically shows results obtained using the system of FIG. 1 or 2;

FIG. 25 is a flowchart of an operating process of the system of FIG. 1 or 2;

FIG. 26 is a flowchart of an operating process of the system of FIG. 1 or 2.

### DETAILED DESCRIPTION OF PREFERRED EMBODIMENTS OF THE INVENTION

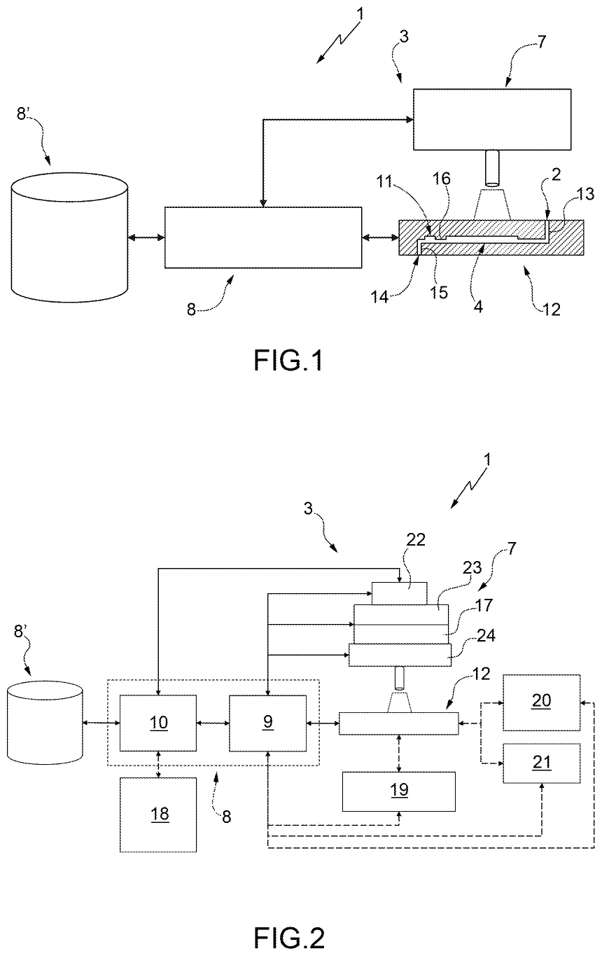

In FIG. 1, in accordance with a first aspect of the present invention, 1 denotes as a whole a microfluidic system for the manipulation (in particular, for the isolation) and/or analysis of particles of a sample. Advantageously, but not necessarily, the microfluidic system 1 is for the manipulation (in particular, for the isolation) of particles of a sample.

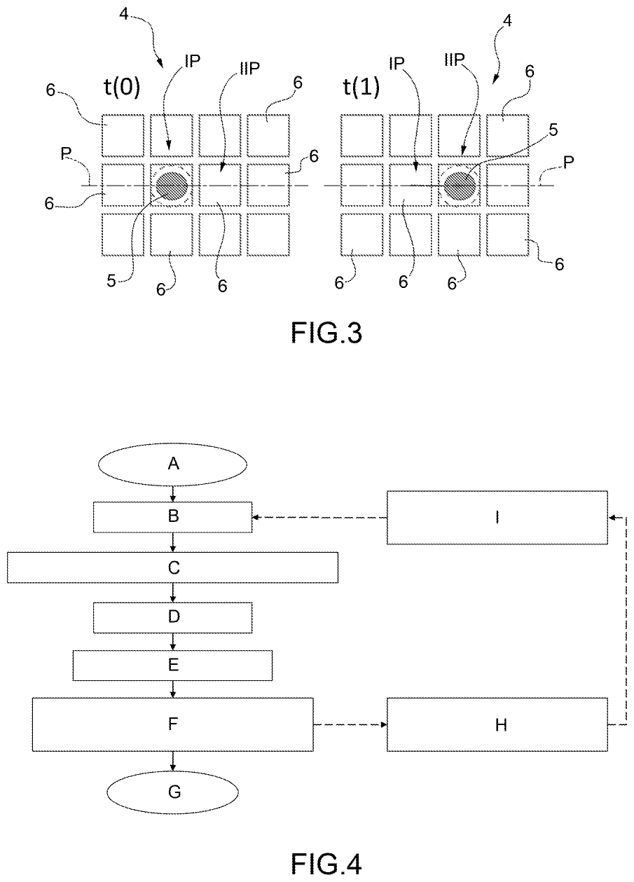

The microfluidic system 1 comprises at least one inlet 2, through which, in use, the sample is inserted in the microfluidic system 1; and a moving assembly 3, which comprises at least one microfluidic chamber 4 and is configured to move at least one specific particle 5 (see e.g. FIG. 3) inside the microfluidic chamber 4.

The moving assembly 3 comprises at least one actuator 6, which is configured to displace the specific particle 5 (and other particles in the sample); a detection device 7 (FIG. 1) which is configured to acquire (at least partial) images of (in particular, of the entire) microfluidic chamber 4; and a control device 8, which is configured to control the actuator 6 (in particular, the actuators 6) so as to move the specific particle 5 (in particular, along a given path P) inside the microfluidic chamber 4.

Images of the microfluidic chamber 4 are defined as the images of the entire microfluidic chamber 4 or of one or more portions of the microfluidic chamber 4.

Note that the path P may have different lengths. For example, the path P may also be the path between two adjacent actuators 6 (and thus extremely short). Alternatively, but not necessarily, the path P extends through a plurality of actuators (e.g., so as to arrive as far as a recovery chamber 11—described further below).

Advantageously, but not necessarily, the moving assembly 3 comprises a plurality of actuators 6 (FIG. 3), which are configured to displace the specific particle 5 (inside the microfluidic chamber 4; in particular, along the path P). In particular, the control device 8 is configured (more precisely but not necessarily, a control unit thereof 9 is configured—FIG. 2) to control the actuators 6 so as to move the specific particle 5 inside the microfluidic chamber 4 (more in particular, along the given path P).

Advantageously, but not necessarily, the moving assembly 3 is configured to move the specific particle 5 (and the other particles of the sample) in a deterministic manner (i.e. in a deliberate manner from an initial given position to a subsequent given position). In particular, the moving assembly 3 is configured to move the specific particle 5 (and the other particles of the sample) in a substantially selective manner relative to the other particles of the sample inside the microfluidic chamber 4.

In particular, the moving assembly 3 is configured (in particular, the actuator (s) is/are configured) to exert a force directly on the specific particle 5 (more in particular, without the force being exerted on the fluid which transfers the movement to the specific particle 5—and to the other particles). For example, each actuator 6 comprises (in particular, is) a respective electrode.

According to some non-limiting embodiments, the moving assembly 3 comprises a displacing system for displacing particles chosen from the group consisting of: travelling waves, thermal flow, local fluid movements generated by electro thermal flow, local fluid movements generated by electro hydrodynamic forces, dielectrophoresis, optical tweezers, opto-electronic tweezers, light-induced dielectrophoresis, magnetophoresis, acoustophoresis (and a combination thereof).

In particular, the displacing system for displacing particles is chosen from the group consisting of: dielectrophoresis, optical tweezers, magnetophoresis, light-induced dielectrophoresis (and a combination thereof). Advantageously, but not necessarily, the displacing system for displacing particles is dielectrophoresis.

According to specific non-limiting embodiments, the moving assembly 3 comprises a dielectrophoresis unit (or system) like for example described in at least one of the patent applications WO-A-0069565, WO-A-2007010367, WO-A-2007049120. More in particular, the moving assembly 3 operates in accordance with what is described in the patent applications with publication number WO2010/106434 and WO2012/085884.

As better shown in FIG. 3, the control device 8 is configured (more precisely but not necessarily, the control unit thereof 9 is configured—FIG. 2) to control the detection device 7 so that the detection device 7 acquires a first image of the aforementioned part of the microfluidic chamber 4 in a first instant t(0), when the specific particle 5 is arranged in a first position IP (of the given path P) inside the part of the microfluidic chamber 4, and a second image of an area of the microfluidic chamber 4 in a second instant t(1) subsequent to the first instant, when the specific particle 5 is arranged in a second position IIP (of the given path P) inside the mentioned area of the microfluidic chamber 4.

In particular, the control device 8 is configured (more precisely but not necessarily, a control unit 9 thereof is configured—FIG. 2) to control the actuators 6 so as to move the specific particle 5 (the specific particles 5) inside the microfluidic chamber 4 from the first position IP to the second position (more in particular, along the path P). More in particular, the first and the second position IP and IIP are intermediate points of the path P.

In other words, the control device 8 is configured (more precisely, but not necessarily, a control unit 9 thereof is configured—FIG. 2) to control the actuators 6 so as to move the specific particle 5 (the specific particles 5) from a start position to an end position of the path P (passing through the first and the second position IP and IIP); where the first and the second position IP and IIP are intermediate points between the start position and the end position.

In some non-limiting cases, the second image is only about an area of the microfluidic chamber 4. Alternatively, the second image is about the entire microfluidic chamber 4. According to some non-limiting embodiments, the first image is only about a part of the microfluidic chamber 4. Alternatively, the first image is about the entire microfluidic chamber 4.

By way of example, FIG. 3 shows the specific particle 5 in the first position IP in the first instant—t(0)—and in the second position IIP in the second instant—t (1).

According to different embodiments, the area of the microfluidic chamber 4 acquired with the second image coincides with or is different from the part of the microfluidic chamber 4 acquired with the first image.

Advantageously but not necessarily, the area of the microfluidic chamber 4 acquired with the second image coincides with the part of the microfluidic chamber 4 acquired with the first image (i.e., the second image is about the part of the microfluidic chamber 4 that is also of the first image).

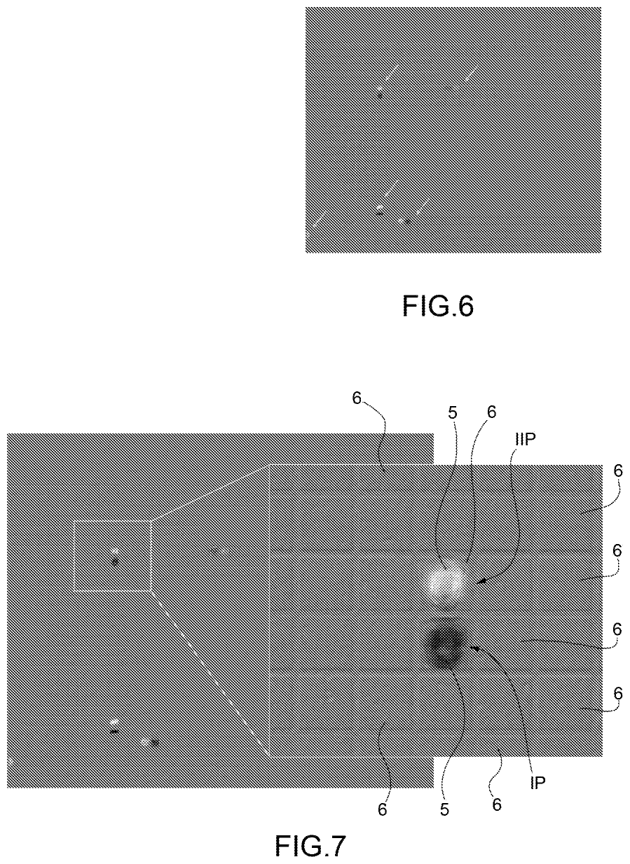

The control device 8 is configured (more precisely, but not necessarily, a process unit thereof 10 is configured—FIG. 2) to process at least one derived image (examples of such a derived image are shown in FIGS. 6, 7, 8, 11 and 12) as a function of (at least) the first image and the second image.

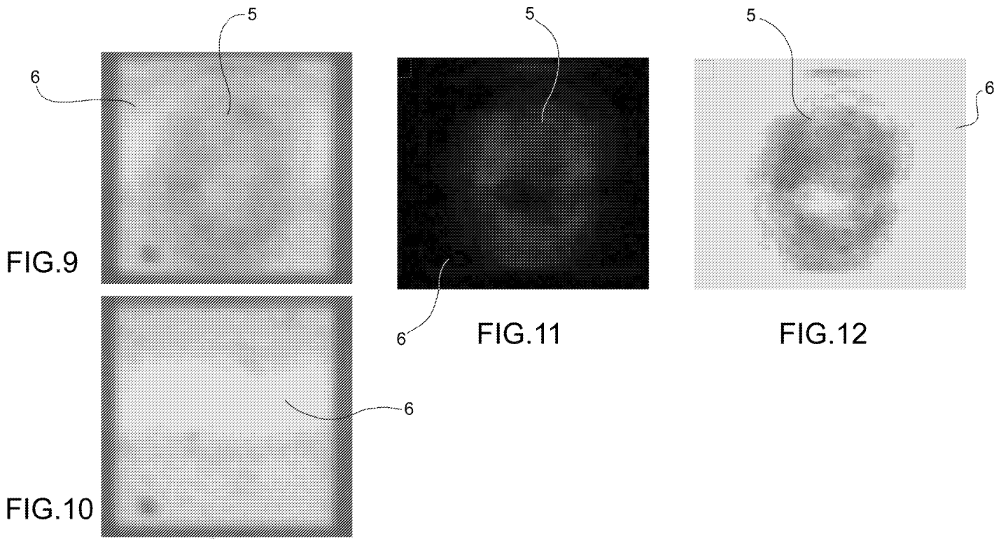

By way of non-limiting example, note that FIG. 5 shows an example photograph taken in the first instant. FIG. 9 is an enlargement of this photograph and shows the first position IP (of the specific particle 5) in the first instant. FIG. 10 is an enlargement of the first position IP of a photograph taken in the second instant. As can easily be seen, the specific particle 5 is arranged in the first instant in the first position IP while in the second instant it is no longer in the first position IP.

By comparing FIG. 5, which is a simple photograph of the part of the microfluidic chamber 4, with FIGS. 6, 7 and 8, which are non-limiting examples of derived images, it is also evident that the particles (and, more precisely, the specific particle 5) are significantly and surprisingly more visible and identifiable thanks to the microfluidic system 1 in accordance with the present invention. Furthermore, thanks to the microfluidic system 1 (and to the method) according to the present invention it is possible to follow the specific particle 5 (each particle) continuously by verifying its position and/or movements throughout the time of interest. It should be noted that, until now, the particles (and their positions) were identifiable with a certain degree of accuracy through detections by fluorescence. These detections are intrinsically discontinuous (after excitation, in just a few instants the particles are no longer visible due to the photochemical degradation phenomenon of the fluorophore) and for some wavelengths (e.g. ultraviolet) are harmful to cells and DNA.

More precisely, it has been experimentally observed that by using the microfluidic system 1 it is surprisingly possible to determine the position and the morphological characteristics of the particles with greater speed, precision and ease. It should be noted, in fact, that not only the particles are highlighted but also the background (and its confusing effect on detection) is practically eliminated, making detection more precise and brighter. Thus, the microfluidic 1 system has, among other things, a reduced risk of losing and/or damaging particles and its operating speed is higher than that of state-of-the-art systems. In this regard, it should be noted that, in order to identify the type and/or group (in particular, type) and/or the position of the particles, it is, among other things, no longer necessary to carry out detections by fluorescence.

Advantageously but not necessarily, the control device 8 is configured (in particular, the process unit thereof 10 is configured) to process the derived image as a function of the difference and/or subtraction between the first image and the second image.

More precisely, but not necessarily, the derived image is the difference and/or subtraction between the first image and the second image.

According to some non-limiting embodiments, the control device 8 is configured to process the derived image as a function the difference between the first image and the second image; in particular, the derived image is the difference between the first image and the second image.

As known is in the field of image processing, subtraction is defined as the superposition of the first image and the inverse (the negative) of the second image. In particular, to perform a subtraction among images, (the value of) each pixel of the second image is subtracted from (the value of) a corresponding pixel of the first image.

Examples of subtraction are shown in FIGS. 6 and 7, wherein the first positions IP of each particle (i.e. the position of each particle in the first instant) are depicted darker and the second positions IIP (i.e. the position of each particle in the second instant) of each particle are depicted lighter.

As is known in the field of image processing, a difference is defined as a subtraction, the result of which is reported as an absolute value. In particular, in order to carry out a difference among images, (the value of) each pixel of the second image is subtracted from (the value of) a corresponding pixel of the first image; the result (value) obtained is reported as an absolute value.

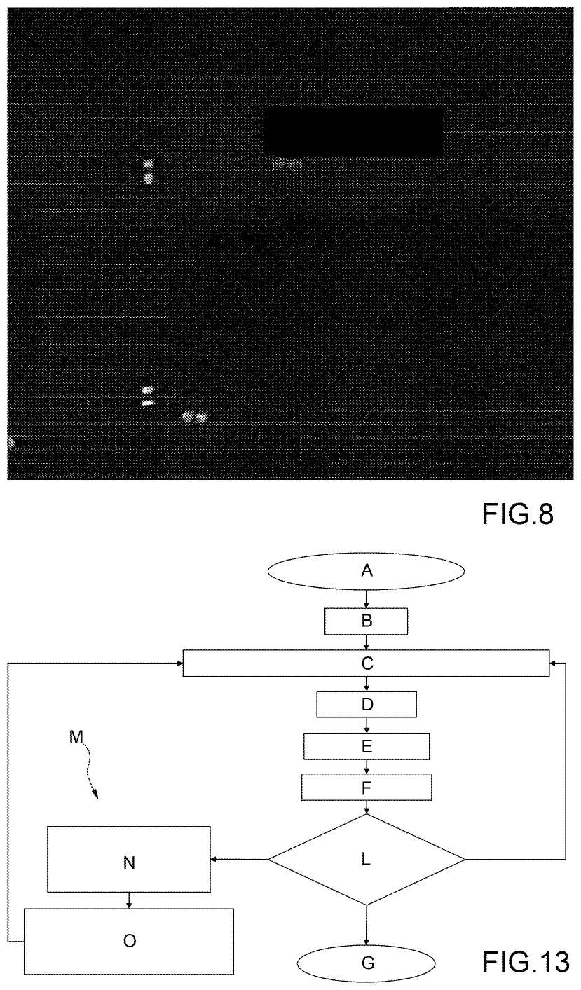

Examples of the difference are shown in FIG. 8, wherein the first positions IP of each particle (i.e. the position of each particle in the first instant) and the second positions IIP (i.e. the position of each particle in the second instant) of each particle are depicted lighter (than the background). FIG. 11 is a detail on an enlarged scale (of a first position IP) of FIG. 8.

Advantageously but not necessarily, the control device 8 is configured (in particular, the process unit thereof 10 is configured) to estimate the second position IIP of the specific particle 5 based on (as a function of) the derived image.

In particular, said second position IIP is different from the first position IP.

Note that in this text estimating refers to measuring (determining, particularly as precisely as possible) something (e.g. the position of the specific particle 5).

Advantageously, but not necessarily, the control device 8 is configured (in particular, the control unit thereof 9 is configured) to control at least the actuator 6 (in particular, the actuators 6) in a third instant, which is subsequent to the first instant and prior to the second instant, so as to move at least the specific particle 5 from the first position IP (in particular, to the second position IIP).

Advantageously, but not necessarily, the moving assembly 3 is configured to exert a force on the specific particle 5 (on the specific particles 5) while the first image and the second image are acquired, in particular, so that the specific particle 5 (the specific particles 5) remains (remain) substantially in the first and, respectively, in the second position IP and IIP.

It has been observed experimentally that, unexpectedly, in this way the first and the second image are of better quality.

More precisely but not necessarily, the control device 8 is configured to control the actuator 6 (in particular, the actuators 6) and the detection device 7 so that the actuator 6 (in particular, the actuators 6) exerts (exert) a force on the specific particle 5 (on the specific particles) while the first image and the second image are acquired by the detection device 7.

Advantageously, but not necessarily, the moving assembly 3 is configured to exert a force on the specific particle 5 (on the specific particles 5) so as to keep the specific particle 5 (the specific particles 5) suspended (them suspended) while the first image and the second image are acquired.

It has been experimentally observed that, surprisingly, in this way the specific particle 5 is made better visible (and therefore the first and the second image and, consequently, also the derived image are of better quality). It was subsequently hypothesised that this is due to the fact that, in this way, the background (more precisely, the base wall of the microfluidic system 1—in particular, of the microfluidic chamber 4) turns out to be out of focus with respect to the particle (s).

More specifically, but not necessarily, the control device 8 is configured to control the actuator 6 (in particular, the actuators 6) and the detection device 7 so that the actuator 6 (in particular, the actuators 6) exerts (exert) a force on the specific particle 5 (on the specific particles) so as to keep the specific particle 5 (the specific particles 5) suspended (them suspended) while the first image and the second image are acquired by the detection device 7.

Where in this text reference is made to one or more particle (s) being “suspended”, it is meant that such particle (s) levitate (s) in (inside) the contained fluid. In other words, the particle (s) is/are kept spaced apart from a base wall of the microfluidic system 1 (in particular, of the microfluidic chamber 4), and optionally, where present, from an upper wall of the microfluidic system 1 (in particular, of the microfluidic chamber 4).

With regard to how to achieve the above, reference is made to the provisions of the aforementioned documents WO-A-0069565, WO-A-2007010367, WO-A-2007049120, WO2010/106434 and WO2012/085884, taking particularly into consideration WO-A-0069565.

In this context, advantageously but not necessarily, the moving assembly 3 comprises an electrode assembly (actuators 6) comprising a first electrode array formed on a support (base wall of the microfluidic chamber 4) and a second electrode array comprising at least one electrode. The second electrode array is turned towards and spaced apart from the first electrode array. The particles (the specific particle (s) 5) and the fluid in which they are immersed (inside the microfluidic chamber 4) are arranged in a region between the first electrode array and the second electrode array. The moving assembly further comprises means for establishing an electric field of constant amplitude on at least one closed imaginary surface located entirely in said fluid. Such means for establishing an electric field of constant amplitude comprise means for applying first periodic signals having a frequency and a first phase to a first sub-set of electrodes of the first electrode array and to the second electrode array and at least another periodic signal having the mentioned frequency and a second phase, opposite to said first phase, to at least another subset of electrodes of the first electrode array.

Referring in particular to FIG. 1, according to some non-limiting embodiments, the moving assembly is 3 configured to transfer at least part of the particles (in particular, including at least the specific particle 5) of a first given type and/or group (in particular, type) of the sample from the microfluidic chamber 4 to a recovery chamber 11 (it also being microfluidic) of the microfluidic system 1 in a substantially selective manner relative to further particles of the sample.

More precisely, but not necessarily, the microfluidic system 1 (more precisely, the moving assembly 3) comprises a microfluidic device 12 (schematically shown in lateral section in FIG. 1), which in turn comprises the microfluidic chamber 4 (and possibly the recovery chamber 11).

According to some non-limiting embodiments, the microfluidic device 12 also comprises a (microfluidic) channel 13, which connects the inlet 2 to the microfluidic chamber, an outlet 14, through which, in use, the specific particle 5 (and/or other particles of interest) can be (is) recovered, a (microfluidic) channel 15 which connects the recovery chamber 11 (arranged between the outlet 14 and the microfluidic chamber 4) to the outlet 14.

In particular, the microfluidic device 12 comprises a channel 16 that connects the microfluidic chamber 4 to the recovery chamber 11.

Advantageously, but not necessarily, the microfluidic device 12 is like the one described in patent applications with publication numbers WO2010/106434 and WO2012/085884 (in these cases, the microfluidic chamber 4 corresponds to the main chamber described therein). In certain non-limiting cases, also the entire microfluidic system 1 is as described in the patent applications with publication numbers WO2010/106434 and WO2012/085884, except as directly indicated in this text.

According to some non-limiting embodiments, the control device 8 is configured (in particular, the control unit thereof 9 is configured) to control at least the actuator 6 (in particular, the actuators 6) so as to move at least the specific particle 5 (and the other particles of the sample) inside the microfluidic chamber 4 (along the given path P) as a function of the data acquired by the detection device 7, more in particular as a function of the aforementioned derived image.

Advantageously, but not necessarily, the microfluidic system 1 comprises a source 17 (in particular, a light source) which is configured to emit at least one given wavelength (in particular, at given wavelengths; in particular, in the visible range).

In particular, the detection device 7 is configured to acquire the first and the second image at least at the given wavelength (in particular, at the given wavelengths; in particular, in the visible range).

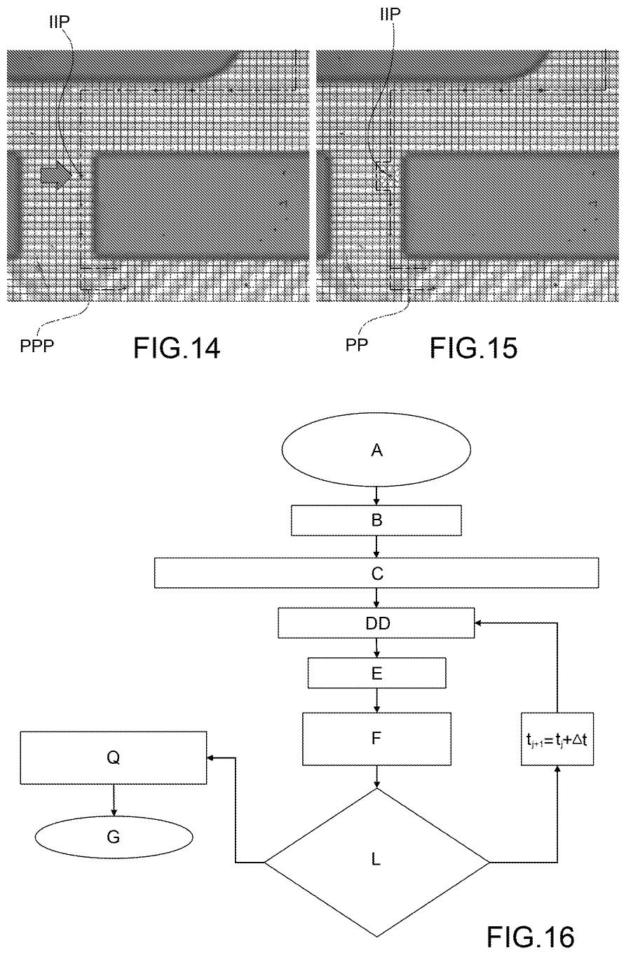

Referring in particular to FIGS. 14 and 15, according to some non-limiting embodiments, the control device 8 is configured (in particular, the process unit thereof 10 is configured) to define at least one further given path PP for at least one further particle of the sample as a function of the derived image. In particular, in such cases, the control device 8 is configured (more in particular, the control unit thereof 9 is configured) to operate at least actuator 6 (in particular, the actuators 6) so that said further particle is moved (and the other particles of the sample are moved) along said further path PP so as not to hit said at least one specific particle 5.

In particular, when the second position IIP coincides with the first position IP (or does not coincide with an expected position), the control device 8 is configured (more in particular, the process unit thereof 10 is configured) to determine the second position IIP as a function of the derived image and to define the further given path PP so that the further given path does not go through the second position IIP.

It was experimentally observed that, in this way, the yield, the efficiency and the operating speed of the microfluidic system were surprisingly improved. In this regard, it should be noted that where the specific particle 5 is blocked inside the microfluidic chamber 4 (or otherwise no longer responds correctly to the controls of the control device 8 through the actuator(s) 6), it is possible to prevent the further particle (or in any case other particles) from being blocked in their movement by the specific particle 5 and/or by a part of the moving assembly 3 that is not functioning correctly in the area of the position IIP and/or

IP. In this regard, it should be noted that it is, for example, possible that an actuator 6 is faulty (or stops functioning correctly); in these cases, in the absence of what is described above, the particles may accumulate in the area of the faulty actuator 6, severely altering the results obtained and/or obtainable from the microfluidic system 1.

By way of example, FIG. 14 shows a hypothesized path PPP previously identified by the control device 8 for the aforementioned further particle. FIG. 15 shows instead the further path PP obtained on the basis of (as function of) the derived image. In particular, in the example shown, the second position IIP is identified as corresponding to the first position IP, and the further path PP (modified with respect to the path PPP) does not pass in the area of the second position IIP.

Advantageously but not necessarily, as for example can be seen from FIG. 15, the path PP is determined by the control device 8 (in particular, by the process unit thereof 10) so that it does not extend even through positions adjacent to the second position IIP.

It has been experimentally observed that, in this way, the performance of the microfluidic system 1 is surprisingly further improved. It is, for example, possible that the problem that has led to the blockage of the specific particle 5 in the position IP may in some way prevent the movements also in neighbouring positions (e.g. when in any case the specific particle has displaced itself slightly, in practice blocking a neighbouring position, as well).

According to some non-limiting embodiments (in particular, when the displacing system of the moving assembly 3 is dielectrophoresis—e.g. as described in WO-A-0069565, WO-A-2007010367 and/or WO-A-2007049120) each position is defined by a respective actuator 6 (e.g. an electrode).

In particular, the control device 8 is configured (in particular, the control unit thereof 9 is configured) to control at least the actuator 6 (more in particular, the actuators 6) so that the further particle follows the further path PP.

Advantageously but not necessarily, the control device 8 is configured (in particular, the process unit thereof 10 is configured) to estimate a detected speed at least of the specific particle 6 (in particular, of the particles) as a function of the derived image based on (as a function of) the distance between the first position IP and the second position IIP and on the time difference between the first instant and the second instant.

According to some non-limiting embodiments, the control device 8 is configured (in particular, the control unit thereof 9 is configured) to control the detection device 7 so that the detection device 7 acquires a plurality of supplementary images of the (part of—or of the entire) microfluidic camera 4 in respective supplementary instants that are subsequent to said first instant (and prior to said second instant). In particular, the supplementary instants are subsequent to one another. More in particular, they are spaced apart from each other by a given time interval Δt (and, even more in particular, constant). Alternatively, the time interval between two supplementary instants can be variable.

Advantageously but not necessarily, the control device 8 is configured (in particular, the process unit thereof 10 is configured) to estimate the time needed by the specific particle 5 to displace itself from the first position IP to the second position IIP on the basis of (as a function of) the supplementary images.

More precisely but not necessarily, the control device 8 is configured (in particular, the process unit thereof 10 is configured) to estimate the second instant when one of the first of the supplementary images (which is thus to be considered as corresponding to the aforementioned second image) shows the specific particle 5 in the second position IIP.

In this way, it has been experimentally observed that it is surprisingly possible to reduce the risk of particles being lost (i.e. not being properly displaced by the actuator (s) 6) inside the microfluidic chamber 4 (along the respective paths P and/or PP) and/or to improve the efficiency and/or the yield of the microfluidic system.

Advantageously but not necessarily, the control device 8 is configured (in particular, the control unit thereof 9 is configured) to operate at least the actuator 6 (in particular, the actuators 6) to displace the specific particle 5 as a function of the detected speed.

In fact, in certain non-limiting cases, for example where the displacing system of the moving assembly 3 is dielectrophoresis (e.g. as described in WO-A-0069565, WO-A-2007010367 and/or WO-A-2007049120), the control device 8 is configured (in particular, the control unit thereof 9 is configured) to activate and deactivate the actuators 6 (arranged along the path P) in sequence as a function of the detected speed.

More precisely, but not necessarily, in use, when the control device 8 (in particular, the process unit thereof 10) estimates that the specific particle 5 has arrived at the first position IP (from a previous position) on the basis of (as a function of) the derived speed, the control device 8 (in particular, the control unit thereof 9) deactivates the actuator 6 (electrode) arranged in the area of the position IP and activates the actuator 6 (electrode) arranged in the second position IIP. In this way, the specific particle 5 displaces itself from the first position IP to the second position IIP.

At this point, when the control device 8 (in particular, the process unit thereof 10) estimates that the specific particle 5 has arrived at the second position IIP on the basis of the derived speed, the control device 8 (in particular, the control unit thereof 9) deactivates the actuator 6 (electrode) arranged in the area of the position IIP and activates the actuator 6 (electrode) arranged in the area of a further position arranged downstream of the second position (along the path P).

Advantageously, but not necessarily, the control device 8 is configured (in particular, the process unit thereof 10 is configured) to determine the type (e.g. whether it is a spermatozoon, a white blood cell, an epithelial cell, a tumour cell, an endothelial cell or a stem cell) of at least the specific particle 5 (in particular each particle) as a function of said derived image.

Alternatively or additionally, the control device 8 is configured (in particular, the process unit thereof 10 is configured) to determine the group of at least the specific particle 5 (in particular, each particle) as a function of said derived image.

In certain non-limiting cases, the control device 8 is configured to identify the type and/or group (in particular, the type) of the specific particle 5 (in particular, using automated supervised non-supervised learning), for example based on reference images (and/or derived image (s)). According to some advantageous but not limiting embodiments, the control device 8 is configured (in particular, the process unit thereof 10 is configured) to extract parameters (in particular, morphological parameters) of at least the specific particle 5 on the basis of (as a function of) the derived image and to determine the type and/or group (in particular, type) of at least one specific particle 5 by using automated learning (in particular, supervised—more in particular, a neural network; or non-supervised—more in particular, clustering).

In particular, the control device 8 is configured (more in particular, the process unit thereof 10 is configured) to determine the respective type and/or group (in particular, type) of each particle of a plurality of particles (of the sample) as a function of the derived image (in particular, on the basis of (as a function of) the—morphological—parameters of each particle obtained from the derived image).

More in particular, the control device 8 is configured (in particular, the process unit thereof 10 is configured) to determine the respective type and/or group (in particular, type) of the specific particle 5 (and possibly of each particle) on the basis of (as a function of) the derived image and of further derived images (obtained in the same manner as the aforementioned derived image—by combining two different images of the microfluidic chamber 4 or of a part thereof taken subsequently).

More details regarding the operation of the control unit 8 (more precisely, of the process unit thereof 10) are given below in relation to the method in accordance with the present invention.

Advantageously, but not necessarily, the microfluidic system 1 comprises a storage unit 8′ (FIG. 1), which is configured to store, for example, what is detected by the detection device 7 and/or what is processed by the control device 8 and/or reference parameters (on the basis of—as a function of—which the type and/or group (in particular, type) of the specific particle 5 and/or the other particles is determined).

The embodiment of the microfluidic system 1 shown in FIG. 2 differs from the microfluidic system 1 of FIG. 1 in that it comprises some further components. For example, it is made explicit in FIG. 2 that the control device 8 comprises the control unit 9 and the process unit 10, which can be separated from each other and (simply) connected or can be fully integrated into a single unit.

In particular, the microfluidic system 1, according to some non-limiting embodiments (FIG. 2), also comprises: an operator interface 18 (HMI—e.g. a screen, a keyboard and/or a pointer—mouse); a temperature control unit 19 for adjusting (maintaining within a desired interval) the temperature of part of (or all of) the microfluidic device 12; a fluidic control device 20 (in particular, controlled by the control device 8) for adjusting the flows of the fluids within the microfluidic device 12; and a recovery unit 21 for collecting the specific particle 5 (and/or other particles) exiting the microfluidic device 12 (in particular, from the outlet thereof 14).

According to some non-limiting embodiments, the detection device 7 (in accordance with what is shown in FIG. 2) comprises: a video camera 22 (in particular, a digital one—or camera); a microscope 23; and the light source 17.

Advantageously but not necessarily, the microfluidic system 1 also comprises a moving device 24, which is configured to move the microfluidic device 12 and/or the detection device 7 relative to each other.

In accordance with a second aspect of the present invention, there is provided a use of the microfluidic system 1 (as defined above) for selectively collecting cells of one or more specific types. For example, there is provided a use of the microfluidic system 1 (as defined above) for (substantially) selectively collecting cells selected from the group consisting of: tumour cells, white blood cells (WBCs), stromal cells, spermatozoas, circulating tumour cells (CTCs), circulating myeloid cells (CMMCs), foetal cells, epithelial cells, erythroblasts, trophoblasts, erythrocytes, endothelial cells, stem cells (and a combination thereof).

In some non-limiting cases, there is provided the use of the microfluidic system 1 (as defined above) for (substantially) selectively collecting cells chosen from the group consisting of: spermatozoa, white blood cells, epithelial cells, tumour cells, endothelial cells, stem cells, foetal cells, nuclei, extracellular vesicles, plant cells (and a combination thereof).

In addition or as an alternative, there is provided a use of the microfluidic system 1 (as defined above) for forensic medicine. In addition or as an alternative, there is provided a use of the microfluidic system 1 (as defined above) for diagnostics (of pathologies—e.g. for tumour diagnosis). In addition or as an alternative, there is provided a use of the microfluidic system 1 for oncology. In addition or as an alternative, there is provided a use of the microfluidic system 1 for prenatal diagnosis.

In the case of a use for oncology, more precisely but not necessarily, there is provided a use for counting and/or the analysis and/or the isolation of Circulating Tumour Cells (CTCs).

In accordance with a third aspect of the present invention, there is provided a method for the manipulation (in particular, for the isolation) and/or analysis of particles of a sample by means of a microfluidic system 1. The microfluidic system 1 comprises at least one inlet 2, through which the sample is inserted in the microfluidic system 1; a moving assembly 3, which comprises at least one microfluidic chamber 4 and is configured to move at least one specific particle 5 inside the microfluidic chamber 4. More precisely, but not necessarily, the moving assembly 3 comprises a microfluidic device 12, which, in turn, comprises the microfluidic chamber 4 (and possibly, a recovery chamber 11, and channels 13, 15 and 16).

Advantageously but not necessarily, the moving assembly 3 further comprises: at least one actuator (e.g. an electrode—in particular, a plurality of actuators), which is configured to displace at least the specific particle 5; a detection device 7 which is configured to acquire images (at least partial images) of the microfluidic chamber 4; and a control device 8, which is configured to control at least one actuator 6 so as to move said at least one specific particle (along a given path P inside the microfluidic chamber 4).

Advantageously, but not necessarily, the microfluidic system 1 is as described above in accordance with the first aspect of the present invention.

The method comprises: a first detection step, during which the detection device 7 acquires a first image of at least a part of the microfluidic chamber in a first instant, when at least the specific particle 5 is arranged in a respective first position IP (in particular, of the given path P) inside the mentioned part of the microfluidic chamber 4; and a second detection step, during which the detection device 7 acquires a second image of at least one area of the microfluidic chamber in a second instant which is subsequent to the first instant, in particular when at least the specific particle 5 is arranged in a respective second position IIP (more in particular, of the given path P) inside the mentioned at least one area of the microfluidic chamber.

In some non-limiting cases, the second image is only about an area of the microfluidic chamber 4. In other words, the second image is a partial image of the microfluidic chamber 4. Alternatively, the second image is about the entire microfluidic chamber 4.

According to some non-limiting embodiments, the first image is only about a part of the microfluidic chamber 4. In other words, the first image is a partial image of the microfluidic chamber 4. Alternatively, the first image is about the entire microfluidic chamber 4.

According to different embodiments, the area of the microfluidic chamber 4 acquired during the second detection step coincides with or is different from the part of the microfluidic chamber 4 acquired during the first detection step. Advantageously but not necessarily, the area of the microfluidic camera 4 acquired during the second detection step coincides with the part of the microfluidic camera 4 acquired during the first detection step (i.e., the first and the second image are about the same part of the microfluidic camera 4).

According to mutually alternative and non-limiting situations, the first position IP and the second position IIP may be different from one another or coincide.

The method further comprises a processing step, during which the control device processes at least one derived image as a function of at least the first image and the second image.

As already indicated above with reference to FIGS. 5 to 12, in this way it has been experimentally observed that the particles (and, more precisely, the specific particle 5) are significantly and surprisingly more visible and identifiable (both as type and/or group (in particular, type) and as position); they can, moreover, be followed continuously (since it is possible to verify their movements and/or position throughout the time span of interest).

Advantageously, but not necessarily, the method also comprises an identification step, during which the control device estimates (i.e. determines as precisely as possible) the second position IIP of at least the specific particle 5 (in particular, of the particles) on the basis of (as a function of) the derived image.

In particular, the second position IIP is different from the first position IP.

Advantageously, but not necessarily, the method further comprises a moving step, during which the control device 8 (in particular, a control unit thereof) controls at least the actuator 6 (in particular, the plurality of actuators 6) in a third instant, which is subsequent to the first instant and prior to the second instant, so as to move at least the specific particle 5 (in particular, to the second position IIP) from the first position IP (in particular, along the given path P).

By way of example, FIG. 3 shows the specific particle 5 in the first position IP in the first instant—t(0)—and in the second position IIP in the second instant—t (1). In particular, during the moving step, the control device 8 controls at least the actuator 6 (more in particular, the actuators 6) to displace the specific particle 5 and a plurality of other particles (more in particular, all the particles present in the microfluidic chamber 4).

More in particular, the control device 8 controls at least the actuator 6 (more in particular, the actuators 6) so as to displace the specific particle 5 and the other particles (even more in particular, all the particles present in the microfluidic chamber 4) in a deterministic manner.

Alternatively or additionally, the control device 8 controls at least the actuator 6 (more in particular, the actuators 6) so as to displace the specific particle 5 and the other particles (more in particular, all the particles in the microfluidic chamber 4) in a substantially selective 20 manner relative to the other particles of the sample inside the microfluidic chamber 4.

Even more precisely but not necessarily, during the moving step substantially all the actuators 6 are activated and deactivated in a coordinated manner in order to substantially displace each particle that is placed substantially in any position of the fluidic chamber (assuming the correct operation of each actuator 6).

Advantageously, but not necessarily, particularly during the moving step, the control device 8 (more precisely, but not necessarily, the control unit thereof 9—FIG. 2) controls the actuators 6 so as to move the specific particle 5 (the specific particles 5) inside the microfluidic chamber 4 along the path P. In particular, the first and the second position IP and IIP are intermediate points of the path P. In other words, the control device 8 (more precisely, but not necessarily, the control unit thereof 9—FIG. 2) controls the actuators 6 so that the actuators themselves move, in particular during the moving step, the specific particle 5 (the specific particles 5) from a start position to an end position of the path P (passing through the first and the second position IP and IIP); where the first and the second position IP and IIP are intermediate points between the start position and the end position.

Advantageously but not necessarily, the moving assembly 3 exerts, in particular during the moving step, a force on the specific particle 5 (on the specific particles 5) while the first image and the second image are acquired (during the first and the second detection step), in particular so that the specific particle 5 (the specific particles 5) remains (remain) (in particular substantially fixed) in the first position IP (during the first detection step) and, respectively, in the second position IIP (during the second detection step).

More precisely but not necessarily, the control device 8 controls the actuator 6 (in particular, the actuators 6) and the detection device 7 so that the actuator 6 (in particular, the actuators 6) exerts (exert) a force on the specific particle 5 (on the specific particles) while the first image and the second image are acquired by the detection device 7, in particular so that the specific particle 5 (the specific particles 5) remains (remain) (in particular substantially fixed) in the first position IP (during the first detection step) and, respectively, in the second position IIP (during the second detection step).

Advantageously, but not necessarily, the moving assembly 3 exerts a force on the specific particle 5 (on the specific particles 5) so as to keep the specific particle 5 (the specific particles 5) suspended (them suspended) while the first image and the second image are acquired (during the first and the second detection step).

More precisely, but not necessarily, the control device 8 controls the actuator 6 (in particular, the actuators 6) and the detection device 7 so that the actuator 6 (in particular, the actuators 6) exerts (exert) a force on the specific particle 5 (on the specific particles) so as to keep the specific particle 5 (the specific particles 5) suspended (them suspended) while the first image and the second image are acquired by the detection device 7.

In this context, according to some non-limiting embodiments, the method provides for manipulating particles immersed in a fluid placed in a region between a first and a second array of electrodes belonging to a group of electrodes. The second electrode array comprises at least one electrode and is facing and spaced apart from the first electrode array. The method provides for applying first periodic signals having a frequency and a first step to a first subset of electrodes of the first electrode array and to the second electrode array and at least a second periodic signal having the mentioned frequency and a second step, which is opposite said first step, to at least another subset of electrodes of the first electrode array, thereby establishing an electric field of constant amplitude on at least one imaginary closed surface arranged entirely in the fluid, whereby the particles are attracted or repelled by a portion of the region enclosed by the at least one imaginary closed surface, depending on the electrical properties of the particles and the fluid.

Advantageously but not necessarily, the first image also contains the other particles in the respective initial positions; the second image also contains the other particles in respective subsequent positions.

FIG. 4 schematically shows a flowchart of a specific and non-limiting example of a procedure implemented in accordance with the aforementioned method for the manipulation (in particular, for the isolation) and/or analysis of particles.

The procedure, advantageously but not necessarily, provides for a start (start—step A); the first detection step (step B); the moving step (step C); the second detection step (step D); the processing step (step E); the identification step (step F); and possibly an end step (end—step G).

Optionally, these steps (more precisely, steps B to F) can be repeated one or more times after the particles 6 have been returned to their original positions (e.g., the specific particle 5 has been returned to the first position IP) (step H) and/or the part and/or the area of the microfluidic chamber 4 that is acquired during the first and the second detection steps is changed (e.g., by displacing the detection device 7 and/or the microfluidic chamber 4) (step I).

Advantageously, but not necessarily (during the moving step), the moving assembly 3 moves (is configured to move) at least the specific particle 5 in a deterministic manner (i.e. in a deliberate manner from an initial given position to a subsequent given position).

In particular (during the moving step), the moving assembly 3 moves (is configured to move) said at least one specific particle in a substantially selective manner relative to (all—to all the) other particles of the sample inside the microfluidic chamber.

For example, the moving assembly 3 exerts a force directly on the specific particle 5 (more in particular, without the force being exerted on the fluid, which transfers the movement to specific particle 5—and to the other particles). In some specific and non-limiting cases, each actuator 6 comprises (in particular, is) a respective electrode.

Advantageously, but not necessarily, the moving assembly 3 is defined as described above in relation to the first aspect of the present invention.

Additionally or alternatively, the control device 8 and/or the detection device 7 and/or the microfluidic device 12 are defined as described above in relation to the first aspect of the present invention.

In particular, (all of) the microfluidic system 1 is defined as described above in relation to the first aspect of the present invention.

Advantageously, but not necessarily, during the processing step, the control device 8 (in particular, a process unit thereof 10) processes the derived image as a function of the difference and/or subtraction between the first image and the second image. In some specific and non-limiting cases, during the processing step, the control device 8 (in particular, a process unit thereof 10) processes the derived image as a function of the difference between the first image and the second image.

In particular, the derived image is the difference (and/or subtraction) between the first image and the second image.

Advantageously, but not necessarily, the processing step comprises an alignment sub-step, during which the first and the second images are aligned (with each other). In such cases, during the processing step, the control device 8 (in particular, a process unit thereof 10) processes the derived image as a function of the (difference and/or subtraction between the) first image and the second image, after the first and the second images have been aligned with each other. Note, that since the first and the second image that were subjected to alignment are a function of the first and of the second image (as acquired), the derived image is also in this case (at least indirectly) a function of the first and of the second image (as acquired).

Thanks to this alignment step, it is possible to obtain brighter derived images and thus reduce the incidence of false positives.

According to some non-limiting embodiments, the alignment sub-step is performed by means of an algorithm of known type, for example Optical Flow or FFT (Fast Fourier Transform).

According to some non-limiting embodiments, the method transfers at least part of the particles (in particular, including at least the specific particle 5) of a first given type and/or group (in particular, type) of the sample from the microfluidic chamber 4 to a recovery chamber 11 (it also being microfluidic) of the microfluidic system 1 (more precisely, of the microfluidic device 12) in a substantially selective manner relative to (all) further particles of the sample.

Advantageously, but not necessarily (during the moving step), the control device 8 (in particular, the control unit thereof 9) controls (is configured to control) at least the actuator 6 (in particular, the actuators 6) so as to move at least the specific particle 5 (in particular, the particles) inside the microfluidic chamber 5 (in particular, along said given path P) as a function of the data acquired by the detection device 7, in particular as a function of the derived image.

According to some non-limiting embodiments, the method comprises an adaptation step, during which the control device 8 defines at least one further given path PP for at least one further specific particle of the sample as a function of the derived image; the moving assembly 3 moves said further specific particle (in particular, the control device 8—more in particular, the control unit 9—operates at least the actuator 6—more in particular, the actuators 6—so that the further specific particle is moved), in particular along the further path PP, so as not to hit said at least one specific particle.

In particular, when the second position IIP coincides with the first position IP or does not coincide with an expected position, the control device 8 (in particular, a process unit thereof) determines the second position IIP as a function of the derived image and defines the further given path PP so that the further given path PP does not go through (in the area of) the second position IIP (and/or through positions near the second position IIP).

According to some non-limiting embodiments, the method comprises a third detection step, during which the detection device 7 acquires a third image of the microfluidic chamber 4 in a further instant subsequent to the second instant, when (at least) the specific particle 6 is arranged in a third position (of the given path P) inside (the part of) the microfluidic chamber 4. The control device 8 traces an actual path followed by at least the specific particle 5 as a function of the derived image and of a further derived image obtained on the basis of (as a function of) the third image and of the second image (e.g. the further derived image is the difference and/or the subtraction of the third image and of the second image).

Advantageously but not necessarily, the further specific particle (and any further particles) is also the subject of the first detection step, the moving step, the second processing step, the identification step (and possibly a verification step as described hereinbelow).

FIG. 13 schematically shows a flowchart of a specific and non-limiting example of a procedure implemented in accordance with the aforementioned method for the manipulation (in particular, for the isolation) and/or analysis of particles.

The procedure provides steps A to G as described above (in particular, with reference to FIG. 4) and a verification step (step L), during which the control device 8 (in particular, the process unit 10) verifies whether the specific particle 5 has moved (and the other particles have moved) correctly.

In particular, if this is the case (i.e. if the control device 8 verifies that the specific particle 5 has moved correctly), the procedure starts again according to a repeatable cycle from the moving step (step C), in other words, the specific particle 5 is moved from the second position IIP (to the aforementioned third position—along the path P) and it is (again) proceeded with the second detection step (D), the processing step (E), the identification step (F) and the verification step (L).

According to some non-limiting embodiments, this cycle is repeated until the specific particle 5 reaches a desired end position and/or the verification step (L) yields a negative result (i.e., following the verification step based on the processing step it is determined that the specific particle 5 has not moved correctly).

In the event that the verification step (L) yields a negative result, in particular the aforementioned adaptation step (step M) is implemented.

In particular, the adaptation step (M) comprises an obstacle creation sub-step (step N), during which the control device 8 creates a (virtual) obstacle in the area of the position where the specific particle 5 is (is blocked); and a redefinition sub-step (step O), during which the further path PP is determined (in particular, during the redefinition sub-step, a respective further path PP is determined for each of the further particles to be moved) which avoids the (virtual) obstacle.

Advantageously, but not necessarily, after the adaptation step, the cycle (steps C to L, in sequence) is repeated (e.g. for the further particle; in particular, for the other particles—the particles that have moved correctly), in particular until the further particle (in particular, each of the other particles) reaches a desired final position and/or the verification step (L) yields a negative result.

Advantageously, but not necessarily, during the first detection step and during the second detection step, the part of the microfluidic chamber 4 and the area of the microfluidic chamber 4, respectively, are lighted with radiations having given wavelengths (in particular, in the visible range).

In particular, the first and the second image are acquired at the aforementioned given wavelengths; more in particular, the first and the second image are acquired in the visible range (even more in particular, they are not acquired at wavelengths outside the visible range).

According to some non-limiting embodiments, the method comprises a speed estimation step, during which the control device 8 estimates a detected speed of at least the specific particle 5 (and of the other particles) as a function of the distance between the first position IP and the second position IIP (in particular, obtained on the basis of—as a function of—the derived image) and the time needed by the specific particle 5 to displace itself from the first position IP to the second position IIP. In particular, the time needed by said at least one specific particle 5 to displace itself from the first position IP to the second position IIP is the difference between said first and said second instant

According to some non-limiting embodiments, the speed estimation step is performed before the specific particle 5 is transferred towards the recovery chamber 11. Alternatively, the speed estimation step is performed while the specific particle is transferred towards the recovery chamber 11.

In particular, the detected speed is estimated as a function of the distance between the first position IP and the second position IIP that are obtained on the basis of (as function of) the derived image and the time between the first and the second instant. Note that, according to some non-limiting embodiments, the distance between the first position IP and the second position IIP corresponds to the distance between two successive actuators 6 (electrodes) (and is, therefore, known).

More in particular, the detected speed is estimated as a function of the distance between the first position IP and the second position IIP, which in turn are estimated on the basis of (as a function of) the derived image obtained as a function of the first and of the second images subjected to alignment (i.e. after the aforementioned alignment sub-step has been performed).

Advantageously, but not necessarily, the image processing step comprise a derived image manipulation step, by which a derived manipulated image is obtained, as a function of which the aforementioned distance between the first position IP and the second position IIP is estimated.

According to some non-limiting embodiments, the image manipulation step comprises a binarisation sub-step, during which each pixel of the derived image is transformed (from grey tones) to black or white (as a function of a threshold grey tone) in order to obtain a binarised derived image.

Alternatively or additionally, the image manipulation step comprises a morphological manipulation sub-step, during which the (advantageously binarised) derived image is subjected to opening, dilating and/or closing operations in order to obtain the manipulated derived image.

During the opening operation, the outermost edges (more precisely, the relative corners) of the representation (s) of the specific particle 5 in the derived image are eroded.

During the dilating operations, the outer edges of the representation (s) of the specific particle 5 in the derived image are dilated.

During the closing operations, the inner edges of the representation (s) of the specific particle 5 in the derived image are dilated. In particular, as a (macroscopic) effect, the closure of any holes inside the image, the filling of any cavities are obtained.

Advantageously, but not necessarily, the distance between the first position IP and the second position IIP is estimated by evaluating the distance between the barycentres (centroids) of the representations (in the first position IP and in the second position IIP) of the specific particle 5 in the derived image (more advantageously, of the manipulated derived image).

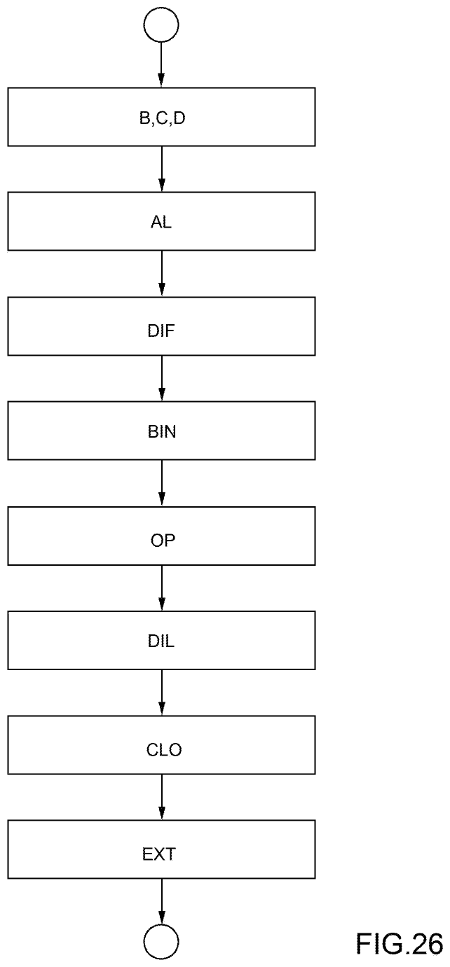

FIG. 26 schematically shows a flowchart of a specific and non-limiting example of an implemented procedure for measuring the distance between the first position IP and the second position IIP.

The procedure provided implementing in succession: the first detection step (step B); the moving step (step C), the second detection step (step D); the alignment sub-step (step AL); a derived image processing (in particular, as a function of the difference and/or subtraction between the first image and the second image—step DIF) the binarisation sub-step (step BIN); the opening operations (step OP); the dilating operations (step DIL); the closing operations (step CLO); an estimation of the distance between the first position IP and the second position IIP (step EXT) on the basis of (as a function of) the manipulated derived image (obtained as a result of the steps: B, C, D, AL, DIF, BIN, OP, DIL and CLO).

For merely explanatory and non-limiting purposes, it should be noted that, in this case, the processing step comprises the steps AL, DIF, BIN, OP, DIL and CLO.

Advantageously, but not necessarily, the method comprises a plurality of supplementary detection steps, during each of which the detection device 7 acquires a respective supplementary image of the microfluidic chamber 4 (in particular, of the aforementioned at least one part of the microfluidic chamber 4; additionally or alternatively, of the aforementioned at least one area of the microfluidic chamber 4) in a respective supplementary instant subsequent to the first instant (and, in particular, prior to said second instant). During the speed estimation step, the time needed by the specific particle 5 to displace itself from the first position IP to the second position IIP is measured on the basis of (as a function of) the supplementary images. In particular, the second instant is estimated when a first one of the supplementary images (which is thus to be considered as corresponding to the aforementioned second image) shows at least the specific particle 5 in the second position IIP.

In particular, the supplementary instants are subsequent to one another (i.e. spaced apart by a given—and constant—time interval Δt). For example, each interval Δt can be from about 5 ms to about 15 ms (in particular, about 10 ms).

FIG. 16 schematically shows a flowchart of a specific and non-limiting example of a procedure implemented in accordance with the aforementioned method for the manipulation (in particular, for the isolation) and/or analysis of particles.

The procedure envisages steps A to C, E, F, G and L as described above (in particular, with reference to FIG. 4) and the supplementary detection step DD. Steps DD, E, F and L, in sequence, are repeated in a cycle at successive supplementary instants (tⱼ+1=tⱼ+Δt) (in particular, spaced apart by the given time interval—Δt) until the specific particle arrives at the second position IIP (and, therefore, the verification step (L) yields a positive result). At this point, the control device 8 estimates the detected speed (step Q).

Advantageously, but not necessarily, the method comprises a conveying step, during which the moving assembly 3 displaces itself (in particular, the control device controls at least the actuator 6—more in particular, the actuators 6—so as to displace) the specific particle 5 (in particular, along the given path P) as a function of the detected speed. This makes it possible to optimise the speed at which each particle moves in a personalised manner.

According to certain non-limiting forms of actuation (during the conveying step), the actuators 6 (electrodes) are activated in succession along the given path P so that, when the specific particle 5 is arranged in the area of a first actuator 6 of the moving assembly 3, the first actuator 6 is deactivated and a second actuator 6 of the moving assembly, which is arranged downstream of the first actuator 6 along the given path P, is activated.

In particular, when the specific particle 5 is arranged in the area of the second actuator 6, the second actuator 6 is deactivated and a third actuator 6 of the moving assembly 3, which is arranged downstream of the second actuator 6 along the given path P, is activated.

More precisely, but not necessarily, the moments in which the first actuator 6 and the second actuator 6 (and possibly the third actuator 6) are activated and deactivated are determined by the control device 8 on the basis of (as a function of) the detected speed.

According to some non-limiting embodiments, the method comprises at least one further first detection step, during which the detection device 7 acquires a further first image (of the aforementioned part) of the microfluidic chamber 4 in a further first instant, when a second specific particle is arranged in a further first position of a second given path inside (the aforementioned part) the microfluidic chamber 4; at least one further second detection step, during which the detection device 7 acquires a further second image (of the aforementioned part) of the microfluidic chamber 4 in a further second instant subsequent to the further first instant, when said further specific particle is arranged in a further second position of the second given path inside (the aforementioned part) the microfluidic chamber 4; a further processing step, during which the control device 8 develops at least one further derived image as a function of said further first image and said further second image (in particular, as a function of the difference and/or subtraction between said further first image and said further second image); and a further speed estimation step, during which the control device 8 estimates a further detected speed of the second specific particle as a function of the distance between the first further position and the second further position, which are obtained on the basis of (as a function of) said further derived image, and of the time needed by the second specific particle to displace itself from the further first position to the further second position.

More precisely, but not necessarily, the method comprises a further conveying step, during which the moving assembly 3 displaces (in particular, said control device 8 controls at least the actuator 6, more in particular the actuators 6, to displace) the second specific particle as a function of said further speed detected along said further given path.

Advantageously but not necessarily, the first detection step coincides with the further first detection step, the second detection step coincides with the further second detection step, the further processing step coincides with said processing step, the further derived image coincides with the aforementioned derived image, the further first image and the further second image coincide with the first image and the said second image, respectively.

In particular, said conveying step and said further conveying step are at least partly simultaneous.

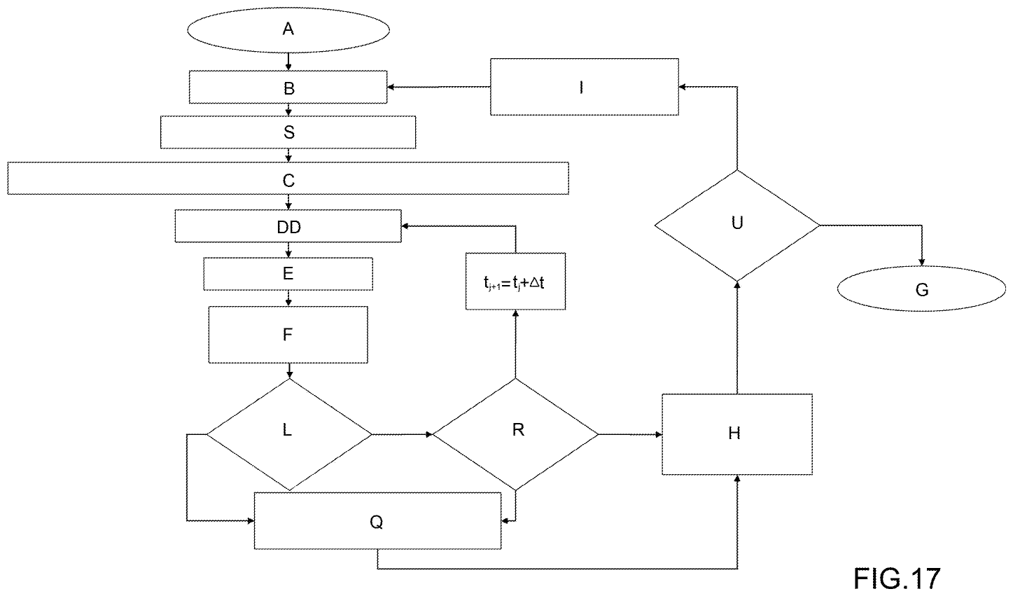

FIG. 17 schematically shows a flowchart of a non-limiting variant of the procedure of FIG. 16. Also in this case, the procedure envisages steps A to C, DD, E, F, G, L and Q. There IS further provided a verification step (step R), during which it is verified whether the total time elapsed since the moving step (C) is higher than a limit time. If this is not the case, a cycle envisaging, in sequence, steps DD, E, F, L and R is repeated. If this the case, this cycle is not repeated and step Q is completed for all the particles.

In particular (as shown in FIG. 17), during the verification step (L), the control device 8 assesses whether all the particles (or supposed particles) have reached their destination. If this is not the case, the verification step (R) is carried out. If this is the case, step Q is completed for all the particles.

Optionally, this procedure also comprises a step for acquiring an image by fluorescence (step S).

Optionally, in the variant of FIG. 17, the procedure envisages a cycle that is repeated (starting from step Q or step R) until during a control step (step U) it is verified that the entire (relevant) microfluidic chamber 4 has been subjected to steps B and DD.

This cycle envisages steps B (possibly S), C, DD, E, F, L, in sequence, and also step H (as described above), subsequent to step Q or to step R and prior to step U; and step I (as described above), subsequent to step U and prior to step B.

In these cases, in other words, steps B, (possibly S) C, DD, E, F, L, Q (and/or R), H, U and I are repeated in a cycle until step U yields a positive result (i.e. when the entire (relevant) microfluidic chamber 4 has been subjected to steps B and DD).

Advantageously, but not necessarily, the method comprises a characterisation step, during which the type and/or group (in particular, type) of at least the specific particle 5 (in particular, of each particle) is determined (in particular, by the control device 8; more in particular, by the control unit 9) as a function of the derived image (in particular, on the basis—as a function—of parameters—e.g. morphological parameters—of said at least one specific particle obtained from said derived image). More precisely, but not necessarily, during said characterisation step, the respective type and/or group (in particular, type) of each particle of a plurality of particles is determined as a function of said derived image (in particular, on the basis—as a function—of parameters—e.g. morphological parameters—of said each particle obtained from said derived image).

It should be noted that in this text, when reference is made to the “characterisation step” or “characterisation”, this means: a classification or grouping.

In particular, “classification” (as used in the sector) is defined as an operation that on the basis of analysis of previously labelled data makes it possible to predict the labelling of future data classes.

In particular, “grouping” (as used in the sector) is defined as an aggregation of unlabelled or unstructured data starting from common characteristics automatically identified by the machine.

In particular, “clustering” (as used in the sector) is defined as a set of multivariate data analysis techniques aimed at selecting and grouping homogeneous elements in a data set.

In particular, “neural network” (as used in the sector) is defined as a computational model composed of artificial “neurons”, which is vaguely inspired by the simplification of a biological neural network. It is therefore a mathematical-computer model consisting of interconnections of information.

In particular, “type” (as used in the sector) is defined as an identification of a data item in a category having a label defined a priori.

In particular, “group” (as used in the sector) is defined as an identification of data as belonging to a category that is recognised starting from common elements without (the need for) a priori identification.

In addition to or as an alternative (to determining the type and/or group (in particular, type) of at least the specific particle 5 as a function of the derived image), during the characterisation step, the type and/or group (in particular, type) of at least the specific particle 5 is determined (in particular, by the control device) as a function of the detected speed (during the aforementioned speed estimation step). In some non-limiting cases, the type and/or group (in particular, type) of at least the specific particle 5 is determined (in particular, by the control device) as a function of a combination of detected speed and derived image and/or morphological parameters.

In particular, when the specific particle 5 is a cell, the viability or integrity of the specific particle 5 (more precisely, if the particle is a living and/or intact or dead and/or apoptotic and/or damaged cell) is determined (in particular, by the control device) as a function of the detected speed (during the aforementioned speed estimation step). More in particular, if the detected speed is below a given threshold speed, the specific particle 5 is considered dead (or apoptotic or damaged); if the detected speed is above a given threshold speed, the specific particle 5 is considered alive and/or intact.

Advantageously but not necessarily, during the characterisation step, the control device 8 determines the type and/or group (in particular, type) of at least the specific particle 5 (in particular, of each particle) using an automated learning (in particular, supervised or non-supervised).

According to some non-limiting embodiments, during the characterisation step, the control device 8 determines the type of at least the specific particle 5 (in particular, of each particle) using a supervised automated learning (in particular, a neural network or convolutional neural network) or the control device 8 determines the belonging group of at least the specific particle 5 (in particular, of each particle) using a non-supervised automated learning (in particular, using the clustering or a non-supervised neural network).

Non-limiting examples of (non-supervised) automated learning are: k-means clustering, DBSCAN clustering, Autoencoder and self-organizing maps.

Non-limiting examples of supervised automated learning are: decision trees, neural networks, convolutional neural networks, support-vector machines, etc.

U.S. Pat. No. 6,463,438 and the article Single-Cell Phenotype Classification Using Deep Convolutional Neural Networks (Oliver Dürr and Beate Sick; Journal of Biomolecular Screening 2016, Vol. 21 (9) 998-1003; DOI 10.1177/187057116631284) describe particle/cell classification/identification systems in more detail.

Advantageously but not necessarily, the characterisation step comprises an extraction sub-step, during which parameters (e.g. morphological parameters) of the specific particle 5 (in particular, of the particles) are extracted (in particular, by the control device 8) on the basis of (as a function of) the derived image; and an identification sub-step, during which the control device 8 determines the type and/or group (in particular, type) of at least the specific particle 5 (in particular, of each particle) on the basis of (as a function of) the extracted (e.g. morphological) parameters.

Advantageously, but not necessarily, the characterisation step of the specific particle 5 (in particular, of the particles) is performed (in particular, by the control device 8) on the basis of (as a function of) the image derived using convolutional neural networks.

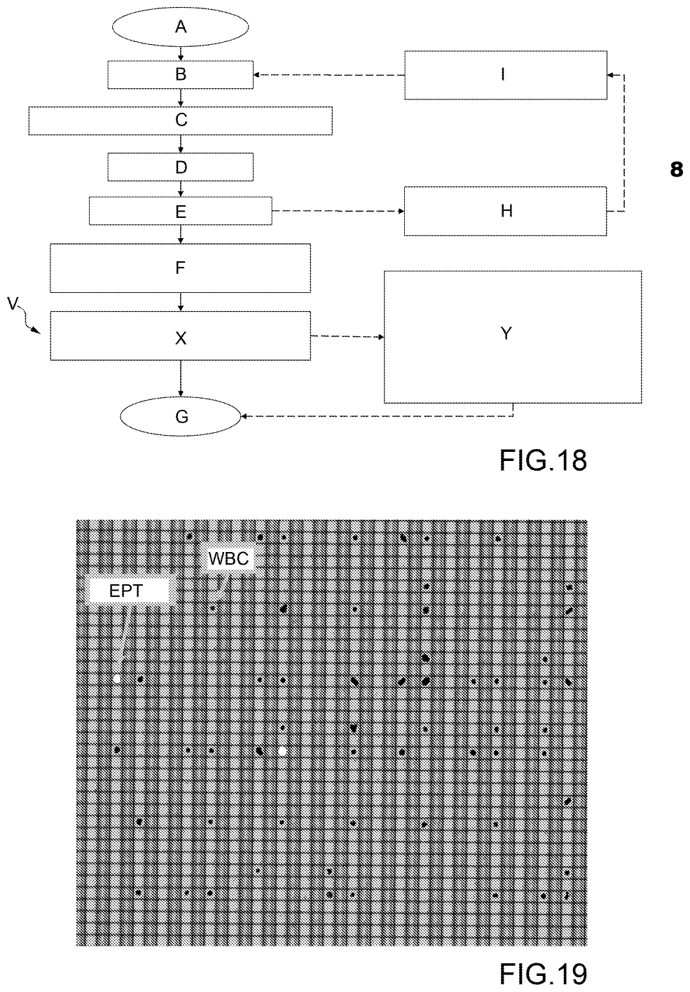

FIG. 18 schematically shows a flowchart of a specific and non-limiting example of a procedure implemented in accordance with the aforementioned method for the manipulation (in particular, for the isolation) and/or analysis of particles.

The procedure envisages steps A to F and G as described above and the extraction sub-step (step X).

Optionally, the procedure comprises the identification sub-step (step Y), between step X and step G to verify if correct in consideration of the figure.

Additionally or alternatively, the procedure comprises steps H and I (and the repetition of the relative cycle, B, C, D, E, H and I in sequence) as described above.

Additionally or alternatively, steps F, X and Y could be performed at least partially simultaneously with steps H and I.

In addition or as an alternative, the loop of steps I and H can start from step D instead of E.

By way of example, FIG. 19 shows how the results obtained experimentally following the method described above are surprisingly reliable, matching the results that were achieved when analysing the sample by carrying out detections by fluorescence. For example, the method described above (in a fully automated way—without the intervention of an operator) has succeeded in, among other things, discriminating between white blood cells (WBC) and epithelial cells (EPT).

Advantageously, but not necessarily, the method comprises at least one re-orientation (e.g., rotation) and/or deformation step, during which the moving assembly 3 re-orients (e.g., rotates) and/or deforms (the actuators 6 are operated—in particular, by the control device 8—so as to re-orient and/or deform) at least the specific particle 5 (the particles) so that at least the specific particle 5 assumes (the particles assume) a different conformation.

More precisely, but not necessarily, the method comprises an additional detection step, during which, the detection device 7 acquires an additional image of the microfluidic chamber 4 (in particular, of the aforementioned at least one part of the microfluidic chamber 4; additionally or alternatively, of the aforementioned at least one area of the microfluidic chamber 4), when at least the specific particle 5 has assumed the different conformation (the particles have assumed respective different conformations). During the processing step, the control device 8 develops an additional derived image as a function of the additional image and one between the first image and the second image (and possibly a further additional image, which is acquired by the detection device 7 prior to the re-orientation and/or deformation step). During the characterisation step, the type and/or group (in particular, type) of at least the specific particle 5 (and also of the other particles) is determined (in particular, by the control device 8) (also) as a function of said additional derived image.

According to some non-limiting embodiments, the additional detection step corresponds to the second detection step and (thus) the additional image corresponds to (is the) aforementioned second image.

Alternatively, the additional detection step is different from the first and the second detection step and (therefore) the additional image does not correspond to (is the) aforementioned second image and/or first image.

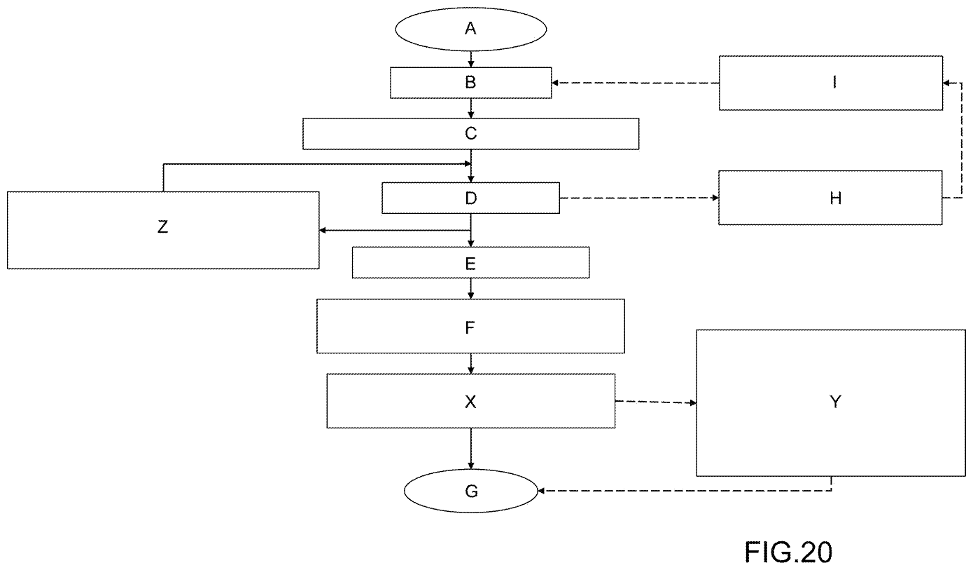

FIG. 20 schematically shows a non-limiting variant of the procedure of FIG. 18 in which the re-orientation step (step Z) is also envisaged (essentially as the only difference). The repetition of the second detection step (D) serves as an additional detection step.

According to some non-limiting embodiments, the method comprises a plurality of further first detection steps, during which the detection device 7 acquires further first images of the microfluidic chamber 4 in further first instants, when specific second particles are arranged in respective further first positions of given second paths inside the microfluidic chamber; a plurality of further second detection steps, during which the detection device 7 acquires further second images of the microfluidic chamber 4 in further second instants subsequent to the further first instant, when the second specific particles are arranged in respective further second positions of the given second paths inside the microfluidic chamber 4; a plurality of further processing steps, during which the control device 8 develops a plurality of further derived images, each, as a function of a respective one of the further first images and a respective one of the further second images (in particular, as a function of the difference and/or subtraction between the respective further first image and the respective further second image); and a characterisation step, during which said specific particle 5 and said second specific particles are classifiably divided into at least two types and/or groups. In particular, the types and/or groups enclose particles with similar characteristics.

Typically, after the characterisation (grouping) step, an operator identifies the different types, dividing the particles, for example, between lymphocytes, platelets, epithelial cells, etc.

Advantageously but not necessarily, the particles are associated with different types (e.g. lymphocytes, circulating tumour cells, epithelial cells, nuclei, etc.) automatically, e.g. during the characterisation (classification) step.

Advantageously, but not necessarily, where there has been previous training, the particles are associated with different types (e.g. lymphocytes, circulating tumour cells, epithelial cells, nuclei, etc.) automatically, e.g. during the characterisation (classification) step.

In some non-limiting cases, the first detection step and the further first detection steps coincide (with each other), the second detection step and the further first detection steps coincide (with each other), the further processing step and the further processing steps coincide (with each other), the further derived images and the aforementioned derived image coincide (with each other), the further first images and the first image coincide (with each other), the further second images and the second image, respectively coincide (with each other).

Advantageously, but not necessarily, the method further comprises a further moving step, during which the control device 8 controls a plurality of actuators 6 (each after a further first detection step and before a further second detection step) in order to move the aforementioned second specific particles 5 from the respective first positions (in particular, to the respective second positions) along said given path P.

According to some non-limiting embodiments, the moving step and the further moving step are (at least partially) simultaneous.

In particular, it should be noted that the further first images include a representation of the second specific particles in the first instant; the further second images include a representation of the second specific particles in the second instant.

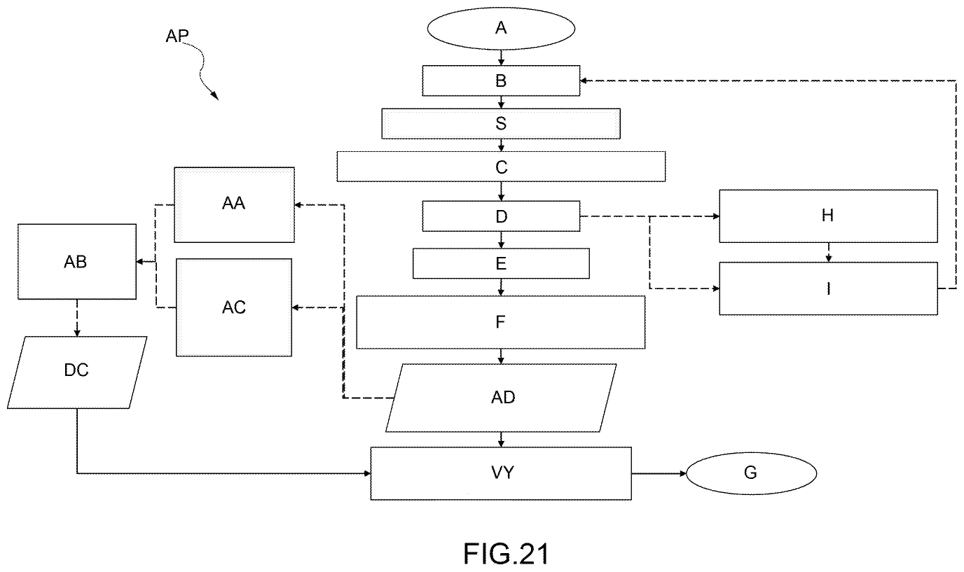

Advantageously but not necessarily, the method comprises a learning step (step AP—FIG. 21) which comprises: at least a first detection sub-step, during which the detection device 7 acquires a first learning image of at least a part of a microfluidic test chamber in a first test instant, when a test particle of a known type is arranged in a first test position (in particular, of a given test path) inside the mentioned part of the microfluidic test chamber; at least one second detection sub-step, during which the detection device 7 acquires a second learning image of an area of the microfluidic test chamber in a second test instant subsequent to the first test instant, when said test particle is arranged in a second test position (in particular, of the given test path) inside the mentioned area of the microfluidic test chamber; and at least one processing sub-step, during which the control device 8 (in particular, the process unit thereof 10) develops a derived test image as a function of the first learning image and of the second learning image and, on the basis of (as a function of) the derived test image, configures (in particular, determines parameters of) an automated learning algorithm (in particular, supervised) in order to train the aforesaid algorithm to identify the (known) type of particles.

In particular, during the characterisation step, the type and/or group (in particular, type) of the specific particle 5 is determined (more precisely, by the control device 8; even more precisely, by the process unit thereof 10) as a function of the derived image using the aforementioned automated learning algorithm.

According to some non-limiting embodiments, during the processing sub-step, the control device 8 (in particular, the process unit thereof 10) extracts (identifies and selects) the parameters of the test particle on the basis (as a function) of (from) the derived test image (step AA) and configures (i.e. trains) the automated learning algorithm with these parameters (step AB), in particular, by correlating them with the known type. For example, step AA is realised using a neural network (particularly convolutional—CNN; or image processing algorithms).

Alternatively or additionally, the parameters of the test particle are morphological parameters (in particular, whose type has been selected by an operator—non-limiting examples of morphological parameters are dimensions, shape, colour etc.). According to some non-limiting embodiments, in such cases, the control device extracts the parameters of the test particle on the basis of (as a function of) the derived test image (step AA) and configures (i.e. trains) the automated learning algorithm with these parameters (step AB), in particular, correlating them with the known type.

For example, step AA is realised using a neural network (particularly convolutional—CNN; or image processing algorithms).

More precisely but not necessarily, the method (in particular, the learning step) comprises a labelling sub-step (step AC), during which an operator determines the correlation between the particle and the type using the available information (for example, the morphological parameters and/or the bright field and/or fluorescence images and/or the fluorescence measurements); during the processing sub-step, the control device 8 (in particular, the process unit thereof 10) extracts (in particular, by image processing) the parameters of the types selected during the selection step from the derived test image (step AA) and configures (i.e. trains) the automated learning algorithm with these parameters (step AB), in particular using the correlation with the known type performed by the operator.

In particular, the second test position is different from the first test position.

In some non-limiting cases, the second learning image is only about an area of the microfluidic test chamber. In other words, the second learning image is a partial image of the microfluidic test chamber. Alternatively, the second learning image is about the entire microfluidic test chamber.

According to some non-limiting embodiments, the first learning image only about a part of the microfluidic test chamber. In other words, the first learning image is a partial image of the microfluidic test chamber. Alternatively, the first learning image is about the entire microfluidic test chamber.

According to different embodiments, the area of the microfluidic test chamber acquired during the second detection sub-step coincides with or is different from the part of the microfluidic test chamber acquired during the first detection sub-step. Advantageously but not necessarily, the area of the test microfluidic chamber acquired during the second detection sub-step coincides with the part of the test microfluidic chamber acquired during the first detection sub-step (i.e. the first and the second learning images are about the same part of the test microfluidic chamber).

Advantageously, but not necessarily, during the first detection sub-step, the detection device 7 acquires the first learning image in the visible range. Alternatively or additionally, during the second detection sub-step, the detection device 7 acquires the second learning image in the visible range.

According to some non-limiting embodiments, the known type is determined (step AC) on the basis of (as a function of) a fluorescence image and/or on the basis of (as a function of) a genetic analysis and/or by an operator on the basis of (as a function of) the derived test image and/or the first learning image and/or the second learning image (acquired in bright field).

Advantageously, but not necessarily, the first detection sub-step, the second detection sub-step and the processing sub-step are repeated a plurality of times, each with a different test particle; more in particular, the first detection sub-step, the second detection sub-step and the processing sub-step are repeated a plurality of times, each with a a test particle of a different known type; in particular, said microfluidic test chamber coincides with the aforementioned microfluidic chamber 4.

According to some non-limiting embodiments, the first and the second detection sub-steps may be coincident for multiple particles.

FIG. 21 schematically shows a flowchart of a specific and non-limiting example of a procedure implemented in accordance with the aforementioned method for the manipulation (in particular, for the isolation) and/or analysis of particles.

In particular, the procedure of FIG. 21 envisages (in addition to steps A, B (possibly S), C, D, E, F, G and the optional H and I with the repetition of the optional cycle B (possibly S), C, D, H and I), a step of selection (crop—AD) of an image of a single particle (in particular, the specific particle 5), steps AA, AC and AB, which involve the definition of correlation data (DC) that relate the derived images, in particular the parameters (e.g. morphological parameters), of the particles with the different types. In particular, the sequence of steps AA, AC and AB are repeated for each particle (of interest), for example during the training step of the algorithm or can be performed only once with all the particles.

According to embodiments, the some non-limiting procedure also provides for a particle characterisation step (step VY—e.g. following the procedure of FIG. 20 or 18). In particular, classification takes place using automated learning algorithms (e.g. supervised, in particular neural networks or convolutional neural networks) configured with the parameters (step DC) generated during the learning step.

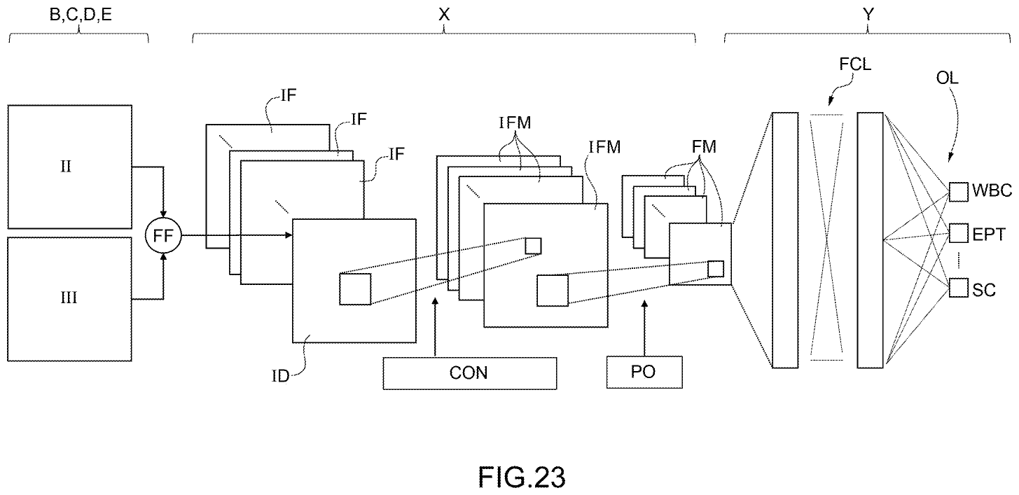

FIG. 23 schematically shows an example of a convolutional neural network (CNN) that can be used during the characterisation step. In this figure, the aforementioned first image is identified with II; the aforementioned second image is identified with III; the (optional) fluorescence images are identified with IF; the derived image is identified with ID; FF denotes the function that combines the first and the second images; IFM denotes the map of the characteristics of the particle (s) obtained by convolution (CON); the selected of maps the characteristics of the particle (s) obtained by reduction (pooling—PO) are identified with FM; FCL denotes completely connected layers; OL denotes the output layer (OL—output layer) that leads to the classification e.g. for white blood cells (WBC), epithelial cells (EPT) and spermatozoa (SC).

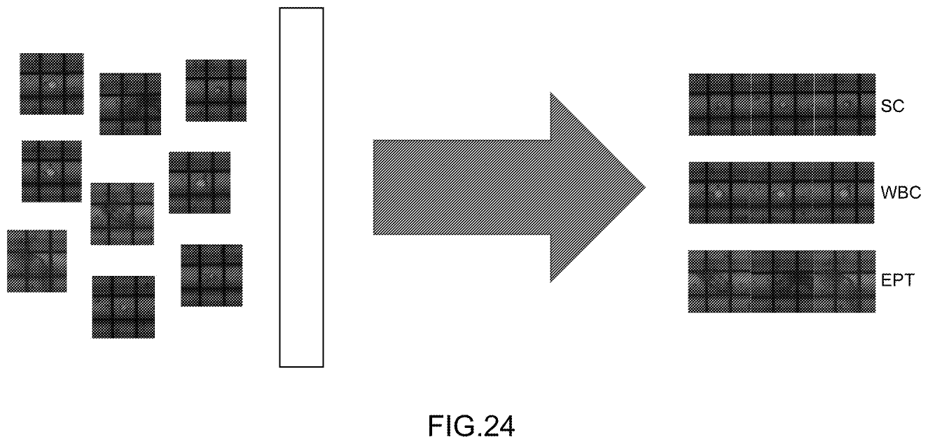

FIG. 24 schematically shows experimental results obtained by the method in accordance with the present invention using a neural network. White blood cells (WBC), epithelial cells (EPT) and spermatozoa (SC) were identified by means of the method. The empty rectangle denotes the processing step (with the processing of the derived image); the arrow denotes the characterisation.

According to some non-limiting embodiments, the method comprises the step of acquiring an image by fluorescence (step S) and a step of identifying the position of all the particles (step AE) on the basis (as a function) of the fluorescence image.

Additionally or alternatively, the method comprises a step of identifying (the position) of all the particles that have moved (step AF—these particles are assumed to be live or intact cells).

Advantageously but not necessarily, the characterisation step (step V) is implemented on the basis (as a function) of what is inferred (also) from the steps of acquisition of a fluorescence image (step S) and of identification of all the particles that have moved (step AF). This makes it possible to identify, among other things, the particles that have not moved.

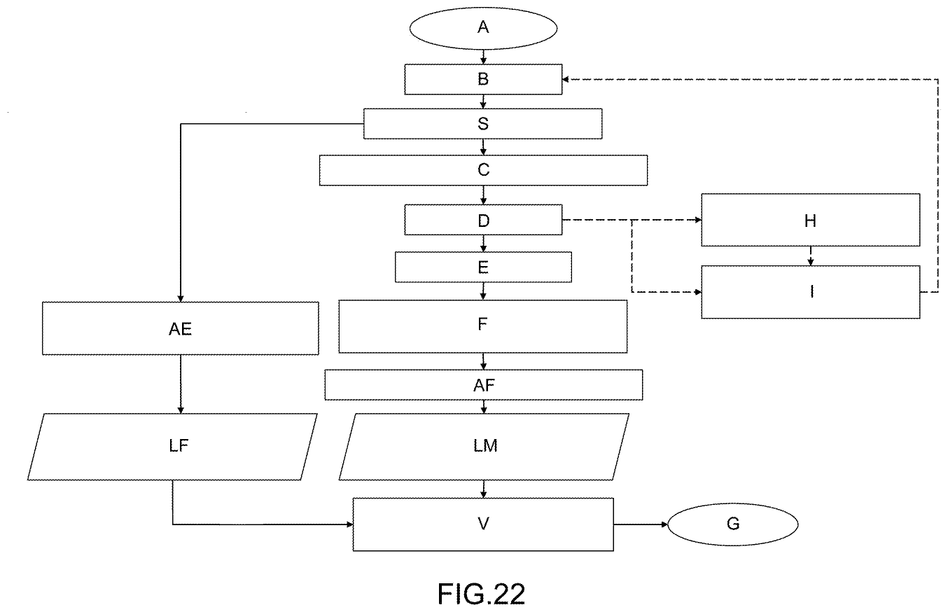

FIG. 22 schematically shows a flowchart of a specific and non-limiting example of a procedure implemented in the accordance with aforementioned method for the manipulation (in particular, for the isolation) and/or analysis of particles. In FIG. 22, the list (of the positions) of the fluorescence-positive cells is denoted by LF and the list (of the positions) of the cells that have moved is denoted by LM.

Advantageously, but not necessarily, the aforementioned speed estimation and classification steps are combined.

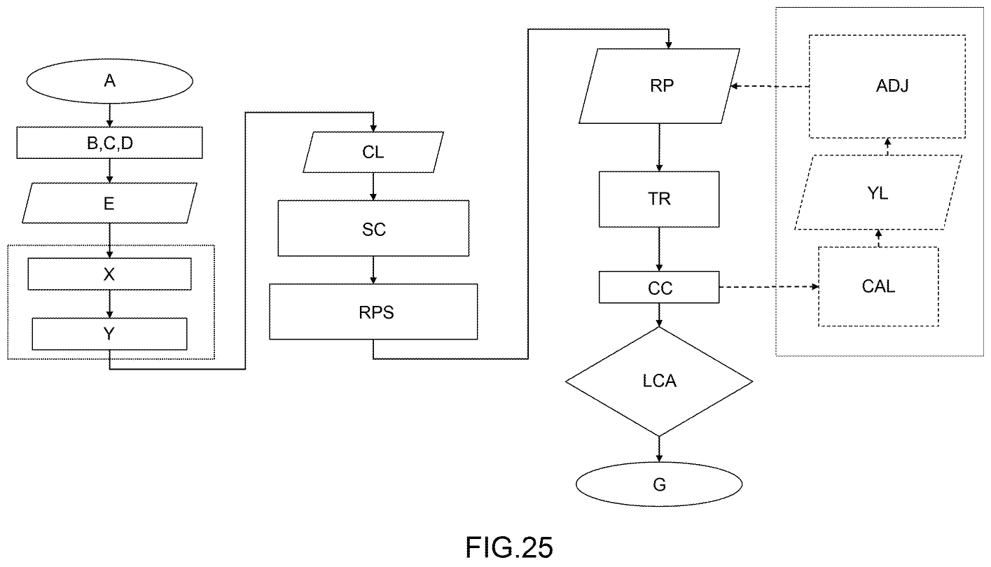

FIG. 25 schematically shows a flowchart of a specific and non-limiting example of a procedure implemented in accordance with the aforementioned method, wherein the speed estimation and classification steps are combined between them.

The method according to this procedure, advantageously but not necessarily, envisages steps A-E, X and Y as defined above to identify the classes (types—CL) of particles and that the speed estimation step (step SC) is implemented for each class. In particular, the method also comprises a parameter identification step (RP—Routing Parameters) for the displacement of the particle (s) (step RPS) as a function of the detected speed; and the aforementioned conveying step (TR).

According to some non-limiting embodiments, it is further provided that a check of the arrival of the particles at the desired position (e.g. in the recovery chamber) (CC step) is carried out and that upon arrival of the last particle (LCA) the procedure ends (step G).

Optionally, an operating cycle can be provided that provides for correcting the parameters (RP—Routing Parameters) for the displacement of the particle (s) (step ADJ) of a given class on the basis (as a function) of the yield for each class (YL) obtained by a calculation (step CAL) as a function of the data detected during the check of the arrival of the particles at the desired position (step CC).

In accordance with a further aspect of the present invention, in addition to or as an alternative to the method referred to in the third aspect of the present invention, there is provided a method for the manipulation (in particular, for the isolation) and/or analysis of particles of a sample by means of a microfluidic system 1 (in particular, as described above in accordance with the first aspect of the present invention).

The microfluidic system 1 comprises at least one inlet 2, through which the sample is inserted in the microfluidic system 1; a moving assembly 3, which comprises at least one microfluidic chamber 4 and is configured to move at least one specific particle 5 inside the microfluidic chamber 4.

More precisely, but not necessarily, the moving assembly 3 comprises a microfluidic device 12, which, in turn, comprises the microfluidic chamber 4 (and possibly, a recovery chamber 11, and channels 13, 15 and 16).

Advantageously but not necessarily, the moving assembly 3 further comprises: at least one actuator 6 (e.g. an electrode—in particular, a plurality of actuators), which is configured to displace at least the specific particle 5; a detection device 7 which is configured to acquire images (at least partial images) of the microfluidic chamber 4; and a control device 8, which is configured to control at least the actuator 6 so as to move said at least one specific particle (along a given path P inside the microfluidic chamber 4).

In particular, the method comprises:
a plurality of the first detection steps, during each of which the detection device 7 acquires a respective first image of a respective part of the microfluidic chamber 4 so that the first images contain a representation of said plurality of particles;
a characterisation step, during which the control device 8 identifies which particles of said plurality of particles are of a given type and/or group as a function of said further first images;
a transfer step, during which at least one particle of the given type and/or group, which has been identified as such during the characterisation step, is transferred by means of the moving assembly 3 (in particular, through operation of the at least one actuator (6); more in particular, of the plurality of actuators) from said microfluidic chamber 4 to a recovery chamber 11 of the microfluidic system 1 in a substantially selective manner relative to further particles of the sample.

Advantageously, but not necessarily, at least part of the characterisation step and at least part of the transfer step take place simultaneously with at least part of the plurality of the first detection steps.

Alternatively or in addition, at least part of the characterisation step and at least part of the transfer step take place before at least part of the plurality of the detection steps.

It has been experimentally observed that in this manner particle recovery takes place in a particularly fast and efficient manner.

According to some non-limiting embodiments, the at least one particle of the given type and/or group is transferred towards the recovery chamber 11 by the moving assembly 3 (in particular, through operation of said at least one actuator 6; more in particular, of said plurality of actuators) during (or before) one of said first detection steps.

Advantageously, but not necessarily, the method comprises a plurality of second detection steps, each of which is subsequent to a respective first detection step and during each of which the detection device 7 acquires a respective second image of the part of the microfluidic chamber 4 acquired during the respective first detection step so that the second images contain a representation of said plurality of particles;
a plurality of moving steps, each of which is subsequent to a respective first detection step and prior to a respective second detection step and during which the control device 8 controls said at least one actuator 6 (in particular, said plurality of actuators) so as to move at least part of said plurality of particles arranged in the area of the part of the microfluidic chamber 4 acquired during the respective first detection step; and a processing step, during which the control device 8 develops a plurality of derived images, each as a function of one of the first images and of a corresponding one of the second images.

In particular, during the characterisation (in particular, classification) step, the control device 8 identifies which particles of said plurality of particles are of a given type and/or group (in particular, type) as a function of said first images.

A second image corresponds to a first image when said second image and said first image are about the same part of the microfluidic chamber 4.

Unless the contrary is explicitly indicated, the content of the references (articles, books, patent applications, etc.) cited in this text is referred to in full herein. In particular, the aforementioned references are incorporated herein by reference.

1. A microfluidic system for the manipulation (in particular, for the isolation) and/or analysis of particles of a sample; the microfluidic system (1) comprising at least one inlet (2), through which, in use, the sample is inserted in the microfluidic system (1); and a moving assembly (3), which comprises at least one microfluidic chamber (4) and is configured to move at least one specific particle (5) inside the microfluidic chamber (4);
the moving assembly (3) comprises: at least one actuator (6) (in particular, a plurality of actuators), which is configured to move said at least one specific particle (5) inside the microfluidic chamber (4); a detection device (7), which is configured to acquire images of the microfluidic chamber (4); and a control device (8), which is configured to control said at least one actuator (6) (in particular, said actuators) so as to move said at least one specific particle (5) along a given path (P) inside said microfluidic chamber (4);
the control device (8) being also configured to control the detection device (7) so that the detection device (7) acquires a first image of at least one part of the microfluidic chamber (4) in a first instant, when said at least one specific particle (5) is arranged in a first position (IP) of the given path (P) inside said at least one part of the microfluidic chamber (4), and a second image of at least one area of the microfluidic chamber (4) in a second instant subsequent to the first instant, when said at least one specific particle (5) is arranged in a second position (IIP) of the given path (P) inside said at least one area of the microfluidic chamber (4);
the control device (8) is configured to develop at least one derived image as a function of said first image and of said second image.

2. The microfluidic system according to claim 1,
wherein the moving assembly (3) is configured to move said at least one specific particle (5) in a deterministic manner;
in particular, the moving assembly (3) is configured to move said at least one specific particle (5) in a substantially selective manner relative to the other particles of the sample inside the microfluidic chamber (4).

3. The microfluidic system according to claim 1 or 2, wherein the control device (8) is configured to develop the derived image as a function of the difference and/or subtraction between said first image and said second image; said control device (8) is configured to control said at least one actuator (6) (in particular, said actuators) in a third instant, which is subsequent to the first instant and prior to the second instant, so as to move said at least one specific particle (5) from said first position (in particular, to said second position); in particular, the derived image is the difference or subtraction between the first image and the second image.

4. The microfluidic system according to any one of the preceding claims, wherein the control device (8) is configured to estimate said second position of the specific particle (5) based on the derived image; said second position being different from said first position.

5. The microfluidic system according to any one of the preceding claims, wherein the moving assembly (3) is configured to transfer at least part of the particles (in particular, including said at least one specific particle) of a first given type and/or group (in particular type) of the sample from said microfluidic chamber (4) to a recovery chamber (11) of the microfluidic system (1) in a substantially selective manner relative to further particles of the sample;
in particular, the control device (8) is configured to control said at least one actuator (6) (in particular, said actuators) so as to move said at least one specific particle (5) along said given path (P) inside said microfluidic chamber (4) as a function of the data acquired by the detection device (7), more in particular as a function of said derived image.

6. The microfluidic system according to any one of the preceding claims and comprising a source (17), which is configured to emit at least one given wavelength (in particular, in the visible range); the detection device (7) being configured to acquire the first image and the second image at said given wavelength (in particular, in the visible range).

7. The microfluidic system according to any one of the preceding claims, wherein the control device (8) is configured to define at least one further given path (PP) for at least one further particle of the sample as a function of the derived image; the control device (8) being configured to operate said at least one actuator (6) (in particular, said actuators) so that said further particle is moved along said further given path (PP) so as not to hit said at least one specific particle (5); in particular, when the second position (IIP) coincides with the first position (IP) or does not coincide with an expected position, the control device (8) is configured to determine the second position (IIP) as a function of the derived image and to define said further given path (PP) so that the further given path (PP) does not go through the second position (IIP).

8. The microfluidic system according to any one of the preceding claims, wherein the control device (8) is configured to estimate a detected speed of said at least one specific particle (5) based on said derived image as a function of the distance between the first position (IP) and the second position (IIP) and on the time difference between the first instant and the second instant.

9. The microfluidic system according to claim 8, wherein the control device (8) is configured to control the detection device (7) so that the detection device (7) acquires a plurality of supplementary images of said microfluidic chamber (4) in respective supplementary instants subsequent to said first instant; the supplementary instants being subsequent to one another (in particular, spaced apart by a given time interval); the control device (8) being configured to estimate the time needed by said at least one specific particle (5) to be moved from the first position (IP) to the second position (IIP) on the basis of said supplementary images.

10. The microfluidic system according to claim 8 or 9, wherein the control device (8) is configured to operate said at least one actuator (6) (in particular, said actuators) so as to move said at least one specific particle (5) as a function of said detected speed.

11. The microfluidic system according to any one of the preceding claims, wherein the control device (8) is configured to determine the type and/or group (in particular, type) of said at least one specific particle (5) as a function of said derived image.

12. The microfluidic system according to any one of the preceding claims, wherein the control device (8) is configured to determine the type and/or group of said specific particle (5) using a supervised or non-supervised automated learning, for example based on reference images.

13. The microfluidic system according to any one of the preceding claims, wherein the control device (8) is configured to extract parameters (in particular, morphological parameters) of said at least one specific particle (5) on the basis of said derived image and to determine the type and/or group (in particular, type) of said at least one specific particle (5) using a supervised or non-supervised automated learning; in particular, the control device (8) is configured to determine the respective type and/or group (in particular, type) of each particle (5) of a plurality of particles as a function of said derived image (more in particular, on the basis of parameters of each one of said plurality of particles obtained from said derived image).

14. The microfluidic system according to any one of the preceding claims, wherein said moving assembly (3) comprises a moving system for moving particles chosen from the group consisting of: travelling waves, thermal flow, local fluid movements generated by electro thermal flow, local fluid movements generated by electro hydrodynamic forces, dielectrophoresis, optical tweezers, opto-electronic tweezers, light-induced dielectrophoresis, acoustophoresis, magnetophoresis and a combination thereof; in particular, the moving system for moving particles is chosen from the group consisting of: dielectrophoresis, optical tweezers, magnetophoresis, light-induced dielectrophoresis and a combination thereof.

15. The microfluidic system according to any one of the preceding claims, wherein the moving assembly (3) (in particular, said at least one actuator (6)) is configured to exert a force directly on said at least one specific particle (5).

16. The microfluidic system according to any one of the preceding claims, wherein the moving assembly (3) is configured to exert a force on said at least one specific particle (5) while said first image and said second image are acquired.

17. The microfluidic system according to claim 16, wherein the moving assembly (3) is configured to exert a force on said at least one specific particle (5) so as to keep said at least one specific particle (5) suspended while said first image and said second image are acquired.

18. A use of the microfluidic system (1) according to any one of the preceding claims, in particular according to claim 11 or 12, to collect in a selective manner cells chosen from the group consisting of: tumour cells, white blood cells, stromal cells, spermatozoa, circulating tumour cells, circulating myeloid cells, nuclei, spores, foetal cells, micro-beads, liposomes, exosomes, extracellular vesicles, epithelial cells, erythroblasts, trophoblasts, erythrocytes and a combination thereof.

19. The use of the microfluidic system (1) according to any one of the preceding claims (in particular, use according to claim 18) for forensic medicine or for prenatal diagnosis or for oncology.

20. A method for the manipulation (in particular, for the isolation) and/or analysis of a sample by means of a microfluidic system (1); the microfluidic system (1) comprises at least one inlet (2), through which the sample is inserted in the microfluidic system (1); a moving assembly (3), which comprises at least one microfluidic chamber (4) and is configured to move at least one specific particle (5) inside the microfluidic chamber (4);
the moving assembly (3) comprises: at least one actuator (6) (in particular, a plurality of actuators), which is configured to move said at least one specific particle (5) inside the microfluidic chamber (4); a detection device (7), which is configured to acquire images of the microfluidic chamber (4); and a control device (8), which is configured to control said at least one actuator (6) (in particular, said plurality of actuators) so as to move said at least one specific particle (5) along a given path (P) inside said microfluidic chamber (4);
the method comprises:
a first detection step, during which said detection device (7) acquires a first image of at least part of the microfluidic chamber (4) in a first instant, when said at least one specific particle (5) is arranged in a respective first position (IP) of the given path (P) inside said at least one part of the microfluidic chamber (4);
a second detection step, during which said detection device (7) acquires a second image of at least one area of the microfluidic chamber (4) in a second instant subsequent to the first instant, when said at least one specific particle (5) is arranged in a respective second position (IIP) of the given path (P) inside said at least one area of the microfluidic chamber (4);
a processing step, during which the control device (8) develops at least one derived image as a function of at least said first image and said second image.

21. The method according to claim 20, and comprising an identification step, during which the control device (8) estimates said second position (IIP) of said at least one specific particle (5) based on the derived image; said second position (IIP) being different from the first position (IP).

22. The method according to claim 20 or 21, wherein the moving assembly (3) moves said at least one specific particle in a deterministic manner; in particular, the moving assembly (3) moves said at least one specific particle (5) in a substantially selective manner relative to other particles of the sample inside the microfluidic chamber (4).

23. The method according to any one of claims 20 to 22, wherein, during the processing step, the control device (8) develops the derived image as a function of the difference and/or subtraction between said first image and said second image;
the method further comprises a moving step, during which said control device (8) controls said at least one actuator (6) (in particular, said plurality of actuators) in a third instant, which is subsequent to the first instant and prior to the second instant, so as to move said at least one specific particle (5) from said first position (IP) (in particular, to said second position) along the given path (P); in particular, the derived image is the difference and/or subtraction between the first image and the second image.

24. The method according to any one of claims 20 to 23, and providing to transfer at least part of the particles (in particular, including said at least one specific particle) of a given type and/or group (in particular, type) of the sample from said microfluidic chamber (4) to a recovery chamber (11) of the microfluidic system (1) in a substantially selective manner relative to further particles of the sample;
in particular, the control device (8) controls said at least one actuator (6) (in particular, said plurality of actuators) so as to move said at least one specific particle (5) along said given path (P) inside said microfluidic chamber (4) as a function of the data acquired by the detection device (7), in particular as a function of said derived image.

25. The method according to any one of claims 20 to 24, wherein, during the first detection step and during the second detection step, said at least one part of the microfluidic chamber (4) and said at least one area of the microfluidic chamber (4), respectively, are lighted with radiations having given wavelengths (in particular, in the visible range); said first and said second image are acquired at said given wavelengths (in particular, in the visible range).

26. The method according to any one of claims 20 to 25, and comprising an adjustment step, during which the control device (8) defines at least one further given path (PP) for at least one further specific particle of the sample as a function of the derived image; the moving assembly (3) moves said further specific particle (in particular, the control device operates said at least one actuator, more in particular said plurality of actuators, so that said further specific particle is moved) along said further given path (PP) so as not to hit said at least one specific particle (5); in particular, when the second position (IIP) coincides with the first position (IP) or does not coincide with an expected position, the control device (8) (in particular, a process unit thereof) determines the second position (IIP) as a function of the derived image and defines said further given path (PP) so that the further given path (PP) does not go through the second position (IIP).

27. The method according to any one of claims 20 to 26, and comprising a speed estimation step, during which the control device (8) estimates a detected speed of said at least one specific particle (5) as a function of the distance between said first position (IP) and said second position (IIP) and on the time needed by said at least one specific particle (5) to be moved from the first position (IP) to the second position (IIP); in particular, the time needed by said at least one specific particle (5) to be moved from the first position (IP) to the second position (IIP) is the difference between said first instant and said second instant; in particular, the detected speed is estimated as a function of the distance between said first position (IP) and said second position (IIP) obtained on the basis of the derived image and of the time between the first and the second instant.

28. The method according to claim 27, and comprising a plurality of supplementary detection steps, during each of which said detection device (7) acquires a respective supplementary image of said microfluidic chamber (4) in a respective supplementary instant subsequent to said first instant; the supplementary instants being subsequent to one another (in particular, spaced apart by a given time interval); during the speed estimation step, the time needed by said at least one specific particle (5) to be moved from the first position (IP) to the second position (IIP) being measured based on said supplementary images.

29. The method according to claim 27 or 28, and comprising a conveying step, during which the moving assembly (3) moves (in particular, said control device controls said at least one actuator—more in particular, said plurality of actuators—so as to move) said at least one specific particle (5) along said given path (P) as a function of said detected speed; in particular, said actuators (6) are operated in succession along the given path (P) so that, when said at least one specific particle (5) is arranged in the area of a first actuator of the moving assembly (3), the first actuator is deactivated and a second actuator of the moving assembly (3), which is arranged downstream of the first actuator along the given path (P) is activated; when said at least one specific particle (5) is arranged in the area of the second actuator (6), the second actuator is deactivated and a third actuator of the moving assembly (3), which is arranged downstream of the second actuator along the given path (P), is activated; the moments in which the first actuator, the second actuator and the third actuator are activated and deactivated are determined by the control device (8) based on said detected speed.

30. The method according to any one of claims 27 to 29 and comprising a characterisation step, during which the type and/or group (in particular, the type) of said at least one specific particle (5) is determined (in particular, by the control device) as a function of the detected speed.

31. The method according to any one of claims 27 to 30 and comprising:
at least one further first detection step, during which said detection device (7) acquires a further first image of the microfluidic chamber (4) in a further first instant, when a second specific particle is arranged in a further first position of a second given path inside the microfluidic chamber;
at least one further second detection step, during which said detection device (7) acquires a further second image of the microfluidic chamber (4) in a further second instant subsequent to the further first instant, when said second specific particle is arranged in a further second position of the second given path inside the microfluidic chamber (4);
a further processing step, during which the control device (8) develops at least one further derived image as a function of said further first image and of said further second image (in particular, as a function of the difference and/or subtraction between said further first image and said further second image); and
a further speed estimation step, during which the control device estimates a further detected speed of the second specific particle as a function of the distance between the first further position and the second further position, obtained based on said further derived image, and on the time needed by the second specific particle to be moved from the further first position to the further second position;
the method further comprises a further conveying step, during which the moving assembly (3) moves (in particular, said control device controls said at least one actuator, more in particular said plurality of actuators, so as to move) said second specific particle as a function of said further detected speed along said second given path;
in particular, the first detection step coincides with the further first detection step, the second detection step coincides with the further second detection step, the further processing step coincides with said processing step, the further derived image coincides with said derived image, the further first image and the further second image coincide with said first image and with said second image, respectively; in particular said conveying step and said further conveying step are at least partially simultaneous.

32. The method according to any one of claims 30 to 31, and comprising a characterisation step, during which the type and/or group (in particular, type) of said at least one specific particle (5) is determined (in particular, by the control device) as a function of said derived image (in particular, on the basis of parameters of said at least one specific particle obtained from said derived image); in particular, during said characterisation step, the respective type and/or group (in particular, type) of each particle of a plurality of particles is determined as a function of said derived image (more in particular, on the basis of parameters of said each particle obtained from said derived image).

33. The method according to claim 32, wherein, during the characterisation said step, control device (8) determines the type and/or group (in particular, type) of said at least one specific particle using an automated learning (in particular, a non-supervised learning or a supervised learning; more in particular, a classification through neural network or a grouping through clustering).

34. The method according to claim 32 or 33, and comprising a learning step, which comprises:
at least one first detection sub-step, during which said detection device (7) acquires a first learning image of at least part of a microfluidic test chamber in a first test instant, when a test particle of a known type is arranged in a first test position inside said at least part of the microfluidic test chamber;
at least one second detection sub-step, during which said detection device (7) acquires a second learning image of at least one area of the microfluidic test chamber in a second test instant subsequent to the first test instant, when said test particle is arranged in a second test position, which is different from the first test position, inside said at least one area of the microfluidic test chamber; and
at least one processing sub-step, during which the control device (8) develops a derived test image as a function of said first learning image and of said second learning image and configures (in particular, determines parameters of) an automated learning algorithm for the identification of the type of particles based on the derived test image;
in particular, the known type is determined on the basis of a fluorescence image and/or on the basis of a genetic analysis and/or by an operator on the basis of the derived test image and/or of the first learning image and/or of the second learning image (acquired in bright field); and/or by the operator on the basis of morphological parameters derived from the derived image; in particular, the first detection sub-step, the second detection sub-step and the processing sub-step are repeated a plurality of times, each with a different test particle; more in particular, the first detection sub-step, the second detection sub-step and the processing sub-step are repeated a plurality of times, each with a test particle of a different known type; in particular, said microfluidic test chamber coincides with said microfluidic chamber (4).

35. The method according to any one of claims 32 to 34 and comprising at least one re-orientation (e.g. rotation) and/or deformation step, during which said moving assembly (e.g. (3) re-orientates rotates) and/or deforms (in particular, said actuators are operated so as to re-orientate and/or deform) said at least one specific particle (5) so that said at least one specific particle (5) assumes a different conformation; an additional detection step, during which said detection device (7) acquires an additional image of the specific particle (5) when said at least one specific particle (5) has assumed said different conformation; during the processing step, the control device (8) develops an additional derived image as a function of said additional image and one between said first image, said second image and a further additional image; during the characterisation step, the type and/or group (in particular, type) of said at least one specific particle (5) is determined (in particular, by the control device) also as a function of said additional derived image.

36. The method according to any one of claims 32 to 35, during wherein, said characterisation step, the respective type and/or group (in particular, type) of each particle of a plurality of particles is determined as a function of said derived image (more in particular, based on parameters of said each particle obtained from said derived image) and at least one particle of a given type and/or group (in particular, type) is identified; the method also comprises a transfer step, during which the at least one particle of a given type and/or group (in particular, type) (in particular, including said at least one specific particle) is transferred (in particular, through operation of said at least one actuator) from said microfluidic chamber (4) to a recovery chamber (11) of the microfluidic system (1) in a substantially selective manner relative to further particles of the sample.

37. The method according to any one of claims 20 to 36, and comprising:
a plurality of further first detection steps, during which said detection device (7) acquires further first images of the microfluidic chamber (4) in further first instants, when a second specific particles are arranged in respective further first positions of second given paths inside the microfluidic chamber;
a plurality of further second detection steps, during which said detection device (7) acquires further second images of the microfluidic chamber (4) in further second instants subsequent to the further first instant, when said second specific particles are arranged in respective further second positions of the second given paths inside the microfluidic chamber (4);
a plurality of further processing steps, during which the control device (8) develops a plurality of further derived images, each as a function of a said further first image and of a said further second image (in particular, as a function of the difference and/or subtraction between a said further first image and a said further second image); and
a characterisation step, during which said specific particle (5) and said second specific particles are divided, in a classified manner, into at least two typological groups.

38. The method according to any one of claims 20 to 37, and comprising a moving step, during which said control device (8) controls said at least one actuator (6) (in particular, said plurality of actuators) in a third instant, which is subsequent to the first instant and prior to the second instant, so as to move said at least one specific particle (5) and a plurality of other particles (in particular, during the moving step, the largest part of the actuators of the moving assembly are controlled so that a plurality of other particles move); said first image also contains said other particles in respective initial positions; said second image also contains said other particles in respective following positions.

39. The method according to any one of claims 20 to 38, wherein the moving assembly (3) is configured to move a plurality of particles inside the microfluidic chamber (4); the control device (8) is configured to control said at least one actuator (6) (in particular, said plurality of actuators) so as to move said plurality of particles inside said microfluidic chamber (4);
the method comprises:
a plurality of the first detection steps, during each of which said detection device (7) acquires a respective first image of a respective part of the microfluidic chamber (4) so that the first images contain a representation of said plurality of particles;
a characterisation step, during which the control device (8) identifies which particles of said plurality of particles are of a given type and/or group as a function of said further first images;
a transfer step, during which at least one particle of the given type and/or group, which was identified as such during the characterisation step, is transferred by means of the moving assembly (3) (in particular, through operation of said at least one actuator (6); more in particular, of said plurality of actuators) from said microfluidic chamber (4) to a recovery chamber (11) of the microfluidic system (1) in a substantially selective manner to relative further particles of the sample;
at least part of the characterisation step and at least part of the transfer step take place simultaneously or before at least part of the plurality of detection steps.

40. The method according to claim 39, wherein the at least one particle of the given type and/or group is transferred towards the recovery chamber by means of the moving assembly (3) (in particular, through operation of said at least one actuator (6); more in particular, of said plurality of actuators) during or before one of said first detection steps.

41. The method according to claim 39 or 40, and comprising:
a plurality of the second detection steps, each of which is subsequent to a respective first detection step and during each of which said detection device (7) acquires a respective second image of the part of the microfluidic chamber (4) acquired during the respective first detection step so that the second images contain a representation of said plurality of particles;
a plurality of moving steps, each of which is subsequent to a respective first detection step and prior to a respective second detection step and during which said control device (8) controls said at least one actuator (6) (in particular, said plurality of actuators) so as to move at least part of said plurality of particles arranged in the area of the part the of the microfluidic chamber (4) acquired during the respective first detection step; and
a processing step, during which the control device (8) develops a plurality of derived images, each as a function of one of the first images and on a corresponding one of the second images;
during said characterisation step, the control device (8) identifies which particles of said plurality of particles are of a given type and/or group as a function of said first images;
a second image corresponds to a first image when said second image and said first image are of the same part of the microfluidic chamber (4).

42. The method according to claim 20 or 41, wherein the moving assembly (3) moves said at least one specific particle (5) in a deterministic manner; in particular, the moving assembly (3) moves said at least one specific particle (5) in a substantially selective manner relative to other particles of the sample inside the microfluidic chamber (4).

43. The method according to any one of claims 20 to 42, wherein the moving assembly (3) exerts a force directly on said at least one specific particle (5), in particular during the moving step.

44. The method according to any one of claims 20 to 43, wherein the moving assembly (3) exerts a force directly on said at least one specific particle (5) during the first and the second detection step.

45. The method according to claim 44, wherein the moving assembly (3) exerts a force directly on said at least one specific particle (5) during the first and the second detection step so as to keep said at least one specific particle (5) suspended while said first image and said second image are acquired.
